(19) United States
(12) Patent Application Publication
(10) Pub. No.: US 2012/0302940 A1
(43) Pub. Date: Nov. 29, 2012

(54) Popcorn Shape Gold Nanoparticle For Targeted Diagnosis, Photothermal Treatment and In-Situ Monitoring Therapy Response for Cancer and Multiple Drug Resistance Bacteria

(75) Inventor: Paresh Chandra Ray, Flowood, MS (US)

(73) Assignee: JACKSON STATE UNIVERSITY, Jackson, MS (US)

(21) Appl. No.: 13/307,652

(22) Filed: Nov. 30, 2011

Related U.S. Application Data
(60) Provisional application No. 61/490,398, filed on May 26, 2011.

(51) Int. Cl. A61M 37/00; C12Q 1/68; A61L 2/02; A23L 3/28; G01N 33/574; G01N 33/569; B82Y 15/00; B82Y 5/00

(52) U.S. Cl. 604/20; 435/7.23; 435/6.14; 435/6.15; 435/7.32; 435/7.35; 422/22; 426/248; 977/795; 977/788; 977/908; 977/904

(57) ABSTRACT

The present invention provides multifunctional popcorn-shaped gold nanomaterial for sensing, treatment and for a unique way of monitoring treatment effectiveness during therapy process of different human cancer cells and pathogenic bacteria. It consists of the following steps 1) Synthesis of popcorn-shaped gold nanoparticles of different sizes. 2) Design of multifunctional popcorn-shaped gold nanoparticle for targeted sensing, therapy and monitoring therapy effectiveness for different human cancer cells (liver, breast, skin and prostate) and drug resistance bacteria. 3) Design of portable SERS sensor for cancer detection, treatment and for monitoring treatment effectiveness using the same instrument. and 4) Application of our approach to detect and kill MDRB from food sample.

### CROSS-REFERENCES TO RELATED APPLICATIONS

This application claims the benefit of U.S. Provisional Application No. 61/490,398 filed May 26, 2011, herein incorporated by reference in entirety.

### STATEMENT REGARDING FEDERALLY SPONSORED RESEARCH OR DEVELOPMENT

This invention was made with government support under NSF-PREM grant #DMR-0611539 awarded by the National Science Foundation. The government has certain rights in the invention

### FIELD OF THE INVENTION

The current application relates to targeted sensing and photothermal treatment, as well as a unique way of monitoring therapy response during the therapy process using multifunctional popcorn shape gold nanoparticle. Specifically, this invention provides popcorn shape gold nanotechnology-driven assay to specifically target and destroy prostate, breast and liver cancer cell and aids in monitoring whether therapy works or not during therapy process. The present invention also demonstrates that this same technology can be used for multiple drug resistance bacteria (MDRB).

### BACKGROUND OF THE INVENTION

Cancer disease and multiple drug resistance bacteria (MDRB) present the greatest challenges in public health care in today's world. Cancer accounted for 13% of all worldwide deaths in 2007, and it is projected to continue rising, with an estimated 12 million deaths in 2030. Current treatment, including surgery, radiation therapy, and chemotherapy, is mostly ineffective against advanced stage prostate cancer disease and is also often associated with severe side effects. As a result, new approaches to treat liver, breast and prostate cancer that do not rely on traditional therapeutic regimes are very urgent for public health, as well as world economy.

For the last two decades, multiple drug resistance bacteria have been a grave public health threat, and a new approach for the treatment that does not rely on traditional antibiotic is urgently needed for public health, as well as world economy.

### SUMMARY OF THE INVENTION

The present invention provides multifunctional popcorn shape gold nanomaterial for sensing, treatment and for a unique way of monitoring treatment effectiveness during the therapy process of different human cancer cells and pathogenic bacteria. It consists of the following steps: 1) Synthesis of popcorn shape gold nanoparticles of different sizes; 2) Design of multifunctional popcorn shape gold nanoparticle for targeted sensing, therapy and monitoring therapy effectiveness for different human cancer cells (liver, breast, skin and prostate) and drug resistance bacteria; 3) Design of portable SERS sensor for detection, treatment and for monitoring treatment effectiveness using the same instrument; 4) Application of our approach to detect and kill MDRB from food sample.

### BRIEF DESCRIPTION OF THE DRAWINGS

Further advantages of the invention will become apparent by reference to the detailed description of preferred embodiments when considered in conjunction with the drawings:

FIG. 1 is a schematic representation of the synthesis of monoclonal anti-PSMA Antibody and A9 RNA Aptamer-Conjugated Popcorn-Shaped Gold Nanoparticles.

FIG. 2 is a schematic representation of the Time-Resolved SERS Setup used for In Situ Measurement of SERS Intensity during Nanotherapy.

FIG. 3 depicts several images used to characterize the nanoparticles.

FIG. 4 depicts several images and graphs of the effects on cancer cells.

FIG. 5 depicts several plots depicting the efficacy of the claimed process.

FIG. 6 is a Schematic representation showing multifunctional nanotechnology-driven approach to selectively target and destroy cancer cell or pathogenic bacteria.

FIG. 7 is Schematic representation showing S-PS8.4 RNA aptamers conjugated popcorn shape gold nanotechnology-driven approach to selectively detect and destroy drug resistant Salmonella bacteria from infected lettuce.

FIG. 8 are photographs showing the colorimetric change upon the addition of drug resistant Salmonella typhimurium DT104 bacteria infected lettuce on the S-PS8.4 RNA aptamers-conjugated popcorn shape gold nanoparticles,

FIG. 9 is a plot showing SERS enhancement (SERS intensity change before and after addition of bacteria) due to the addition of different bacteria to S-PS8.4 RNA aptamers-conjugated popcorn shape gold nanoparticles

FIG. 10 is TEM image showing S-PS8.4 RNA aptamers-conjugated popcorn shape gold nanoparticles before the addition of drug resistant Salmonella typhimurium bacteria.

FIG. 11 is TEM image demonstrating aggregation of S-PS8.4 RNA aptamers-conjugated popcorn shape gold nanoparticles after the addition of 10³ cfu/mL drug resistant Salmonella typhimurium bacteria

FIG. 12 is TEM image demonstrating aggregation of S-PS8.4 RNA aptamers-conjugated popcorn shape gold nanoparticles as well as formation of microbial clusters in presence of 10⁴ cfu/mL drug resistant Salmonella typhimurium bacteria.

FIG. 13 TEM image demonstrating aggregation of S-PS8.4 RNA aptamers-conjugated popcorn shape gold nanoparticles as well as formation of microbial clusters in presence of 10⁵ cfu/mL drug resistant Salmonella typhimurium bacteria

FIG. 14 is a TEM image showing aggregation of S-PS8.4 RNA aptamers-conjugated popcorn shape gold nanoparticles as well as formation of microbial clusters in presence of 10⁶ cfu/mL drug resistant Salmonella typhimurium bacteria

FIG. 15 is a TEM image of E-Coli bacteria in presence S-PS8.4 RNA aptamers-conjugated popcorn shape gold nanoparticles

FIG. 16 is Bright field inverted microscope images of multifunctional popcorn shape gold nanoparticle conjugated HEPG2 lever cancer cells, after therapy for 30 minutes and after stained with Trypan Blue

FIG. 17 are plots demonstrating popcorn shape gold nanotechnology base photothermal therapy for selective killing of cancer cell

FIG. 18 show Colonies of drug resistant Salmonella typhimurium bacteria before and after exposing infected lettuce with S-PS8.4 RNA aptamers-conjugated popcorn shape gold nanoparticle using 670 nm light for 20 minutes.

FIG. 19 is a TEM image demonstrating irreparable damage of cluster of bacterial cell surfaces when S-PS8.4 RNA aptamers-conjugated popcorn shape gold nanoparticle conjugated Salmonella typhimurium bacteria were exposed to 676 nm NIR radiation for 20 minutes.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

We disclose herein protocols for the controlled preparation of popcorn shape gold nanostructures and development of multifunctional popcorn shape gold nanomaterials for imaging, photothermal therapy and to monitor therapy effectiveness during therapy process.

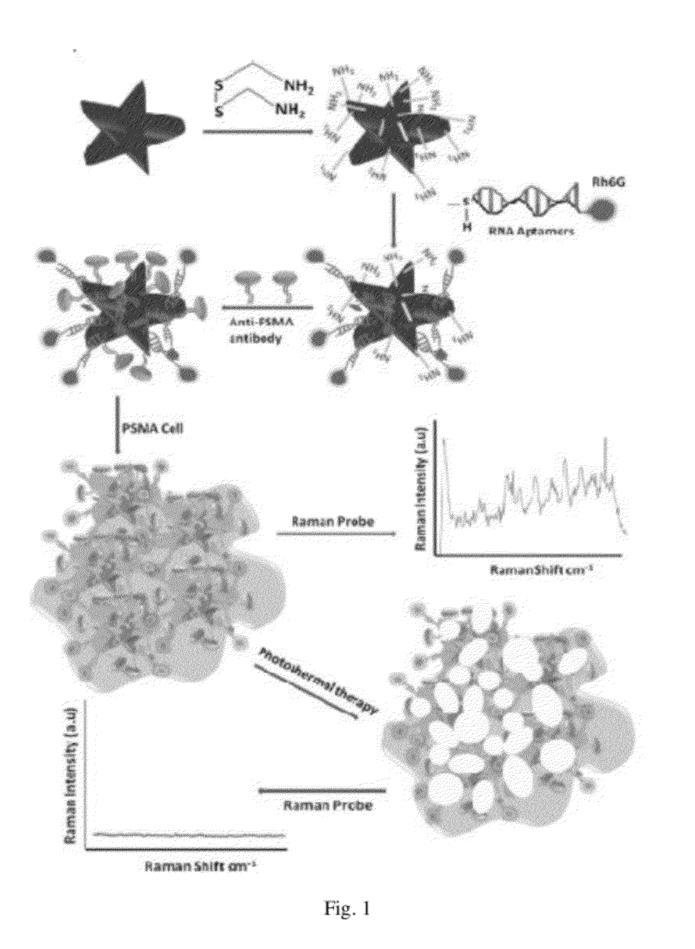

As will be appreciated from FIG. 1, in nano-popcorn, the central sphere acts as an electron reservoir, while the tips are capable of focusing the field at their apexes and provide sufficient field of enhancement. As a result, the low cross-section Raman signals are amplified several orders of magnitude, particularly in narrow nanoscaled corners and edges. This very high sensitivity afforded by surface enhanced Raman spectroscopy (SERS), along with the highly informative spectra characteristic of Raman spectroscopy, makes nano-popcorn based SERS unique for ultrasensitive biological analysis. We have demonstrated multifunctional popcorn shape gold nanotechnology-driven SERS assay for targeted sensing, nanotherapy treatment and in-situ monitoring photothermal nanotherapy response during the therapy process of prostate cancer cell. We further disclose that multifunctional popcorn shape gold nanotechnology-driven approach can be used for targeted detection, therapy and for monitoring therapy effectiveness for breast and liver cancer cells and as well as pathogenic bacteria, such as drug resistant Salmonella.

Furthermore, our nanotechnology-based assay has an enormous potential for providing effective, noninvasive treatment of cancer and drug resistant bacteria in vivo via photothermal therapy.

Sensing, Targeting, and Therapy for Cancer Cells Using Popcorn Shape Gold Nanoparticle

The human prostate cancer cell line, LNCaP, expresses a high level of prostate-specific membrane antigen (PSMA) relative to normal cells of the prostate. It has been shown that PSMA expression increases with clinical stage. There is mounting evidence that normal tissue, including epithelium of the duodenum, kidney, endometrium, and breast, also expresses PSMA. As a result, immunophenotypic analyses of cancer cells using antibody probes for specific surface antigens can dramatically influence selectivity and lead to false positive signals.

Target cell-specific aptamers have the potential to serve as molecular probes for specific recognition of the cancerous cells, but unfortunately, aptamers have weak binding affinity and thus give low signal in molecular imaging, limiting their ability for highly sensitive detection of cancer cells. Also, during the early stages of cancer development, cancer cells will have a very low density of target membrane proteins for recognition of specific cancer cells. As a result, single-aptamer/antibody binding will not be enough to detect early-stage cancer development, and multivalent binding is usually considered to be essential for early-stage disease diagnostics.

For selective sensing, therapy, and monitoring of therapy progress, we disclose a method of conjugating gold nano-popcorn with multiple PSMA-specific targets: anti-PSMA antibody and Raman dye (Rh6G) attached to A9 RNA anti-PSMA aptamers. Rh6G-modified RNA aptamers covalently attached to the surface serve a dual function as targeting molecules and Raman dye-carrying vehicles.

Materials and Experiments

Hydrogen tetrachloroaurate (HAuCl4.3H2O), NaBH4, sodium citrate, cystamine dihydrochloride, and cetyl trimethylammonium bromide (CTAB) were purchased from Sigma-Aldrich and used without further purification. Monoclonal anti-PSMA antibody was purchased from Thermo Fisher Scientific, and 3′-SH— and 5′—Rh6G modified A9 RNA aptamers were purchased from Midland Certified Reagent. The human prostate cancer cell line LNCaP, which overexpresses a high level of PSMA, was obtained from the American Type Culture Collection (ATCC, Rockville, Md.). PSMA negative human prostate cancer cell line (PC-3) was also purchased from ATCC. Human skin HaCaT keratinocytes, a transformed human epidermal cell line, was obtained from Dr. Norbert Fusenig of the Germany Cancer Research Center (Heidelberg, Germany).

Synthesis of Popocorn-Shaped Gold Nanoparticles.

Our gold nano-popcorn synthesis was achieved through a two-step process, using seed-mediated growth. In the first step, very small, reasonably uniform, spherical seed particles were generated using trisodium citrate as stabilizer and sodium borohydride as strong nucleating agent. In the second step, we used ascorbic acid as weak reductant, as well as CTAB as shape-templating surfactant, so that the seeds grew into larger particles of the particular morphology we desired.

The ascorbic anions transfer electrons to the seed particles, which reduce gold ions to form a gold shell, which grows into different shapes in the presence of CTAB. Spherical gold seeds were synthesized by mixing aqueous solutions of hydrogen tetrachloroaurate (III) hydrate with trisodium citrate in 20 mL of double distilled deionized water (18 MΩ), where the final concentration of HAuCl4.3H2O was 2.5×10⁻⁴ M, and the concentration of trisodium citrate was 10⁻⁴ M. An ice-cooled, freshly prepared aqueous solution of sodium borohydride, NaBH4 (0.1 M, 60 μL), was then added under vigorous stirring. The solution turned pink immediately after the addition of NaBH4 and became red after it was kept in the dark overnight. The nanoseeds exhibited absorption spectra with a maximum at 510 nm, which corresponds to a 4.3 nm seed, which was confirmed by TEM.

Subsequently, nano-popcorn was synthesized using the seed-mediated growth procedure in the presence of CTAB. For this preparation, we dissolved 0.05 g of CTAB in 46.88 mL of H2O by sonication in a small vial, and then we added 2 mL of 0.01 M HAuCl4.3H2O under constant stirring. Next, 0.3 mL of 0.01 M AgNO3 was added to the solution to mix properly. After that, we added 0.32 mL of 0.1 M ascorbic acid dropwise as a reducing agent. The solution turned from yellow to colorless. To this colorless solution, we instantly added 0.5 mL of gold nano-popcorn seed and mixed the solution for 2 minutes. The color changed immediately and became blue within 2 minutes, indicating the formation of popcorn nanostructures.

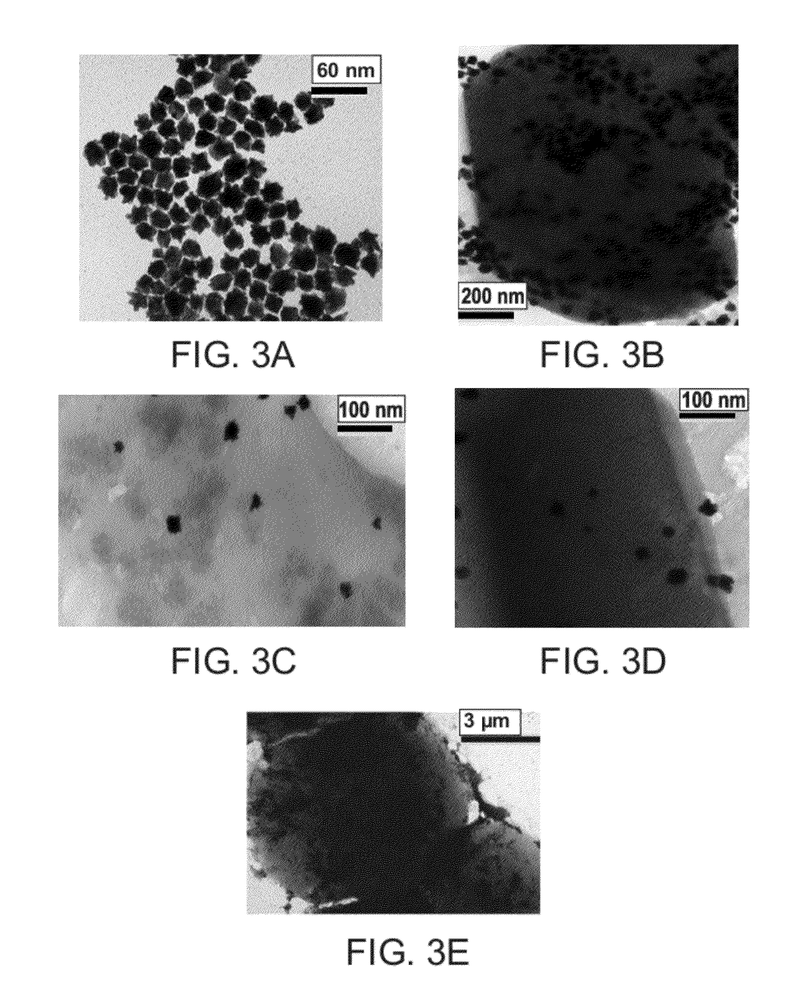

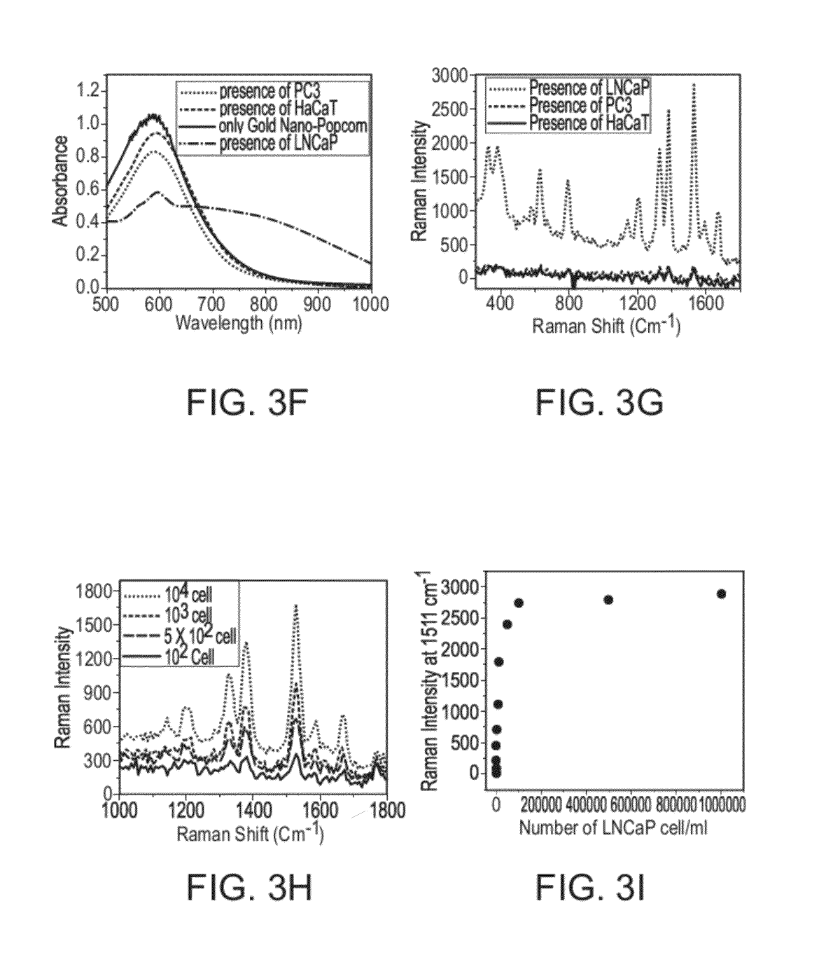

Transmission electron microscopy (TEM, JEM-2100F instrument) and UV-visible absorption spectroscopy were used to characterize the nanoparticles (as shown in FIG. 3, below). A popcorn-shaped gold nanoparticle has only one plasmon bond, like spherical gold nanoparticles, but its λ max is shifted about 60 nm in comparison to that of a spherical gold nanoparticle of the same size. The popcorn-shaped gold nanoparticles concentration was measured using the plasmon absorption peak at 580 nm, given that the popcorn-shaped nanoparticles extinction coefficient is 4.6×10⁹ M-1 cm⁻¹. The extinction coefficient was measured by using ICP analysis to quantitatively determine the gold concentration in nanoparticle solution and nanoparticle volume measured by TEM. This experiment was performed 5-6 times, and average values are reported here.

Preparation of Multifunctional Popcorn-Shaped Nanoconjugates.

As discussed above, popcorn-shaped gold nanoparticles were synthesized using a seed-mediated growth procedure in the presence of CTAB. The above procedure produced popcorn-shaped gold nanoparticles with CTAB coating. CTAB is known to be cytotoxic, and, as a result, it will not be ideal for in vivo diagnosis. Furthermore, since CTAB is positively charged at physiological pH, it will be able to attract negatively charged proteins easily. Thus, CTAB-coated popcorn-shaped gold nanoparticles face severe nonspecific binding problems. To overcome this, we modified the oval-shaped gold nanoparticle surface with -3′-SH— and 5′-Rh6G-modified A9 RNA aptamer capture oligonucleotides, A9 RNA aptamers, and cystamine dihydrochloride (as shown in FIG. 1) using known methods (see References 26, 40, 50 below). —SH-labeled RNA capture oligonucleotides and A9 RNA aptamers were gradually exposed to gold nanomaterial in the presence of 0.1 M NaCl in a PBS buffer over a 16-hour period according to procedures as disclosed in References 26, 40, and 50.

To remove the unbound RNA, we centrifuged the solution at 13,000 rpm for 20 minutes, and the precipitate was redispersed in 2 mL of the buffer solution. We repeated this process three times. To measure the number of aptamer molecules in each gold nanoparticle, after conjugation, we treated the aptamer-conjugated gold nanoparticles with 10 μM potassium cyanide to oxidize the gold nanoparticles. After that, the solution containing the released Rh6G-labeled aptamers was collected for the fluorescence analyses. The amount of Rh6G-labeled aptamers was measured by fluorescence. By dividing the total number of Rh6G-labeled aptamers by the total number of nanoparticles, we estimated that there were about 400-500 aptamers per popcorn-shaped gold nanoparticle. This experiment was performed 5-6 times, and average values are reported here.

To modify the gold nanoparticle surface with amine groups (as shown in FIG. 1), we added 30 mM cystamine dihydrochloride to 50 mL of gold nanoparticle, and the solution was kept at 50° C. for several hours under constant sonication. After that, the excess cystamine dihydrochloride was removed by centrifugation at 8000 rpm for several minutes. For covalent immobilization of the monoclonal anti-PSMA antibody onto the amine, we used the well-established glutaraldehyde spacer method. To remove the excess antibody, we washed the aptamer- and anti-PSMA antibody-conjugated nanoparticles several times with PBS. To measure the number of anti-PSMA antibody molecules in each gold nanoparticle, we performed the above-described process with Rh6G-labeled anti-PSMA antibody. After conjugation, we performed exactly the same process that we did for Rh6G-labeled aptamers. We estimated that there were about 80-100 anti-PSMA antibodies per popcorn-shaped gold nanoparticle. This experiment was performed 5-6 times, and average values are reported below. During the aptamer conjugation and immobilization of the antibody, we did not note any aggregation of gold nanoparticles as examined by TEM and UV-visible absorption spectroscopy.

Characterization of Multifunctional Gold Nanoparticles.

To characterize popcorn-shaped gold nanoparticle conjugates with A9 aptamer and anti-PSMA antibody, we performed DLS measurements as shown in Table 1. The DLS measurements were performed using a Malvern Zetasizer Nano instrument.

TABLE 1
Size of Popcorn-Shape Gold Nanoparticles before and after
Conjugation with Aptamer and Antibody

| | hydrodynamic |
|---|---|
| material | diameter (nm) |
| only popcorn-shaped gold nanoparticles | 28 ± 2 |
| A9 aptamer-conjugated popcorn-shaped gold | 40 ± 2 |
| nanoparticles | |
| anti-PSMA antibody-conjugated popcorn-shaped gold | 44 ± 2 |
| nanoparticles | |
| multifunctional popcorn-shaped gold nanoparticles | 43 ± 3 |

As shown in Table 1, popcorn-shaped gold nanoparticles have an average size of about 28 nm, which can be seen clearly from our TEM data. The addition of A9 aptamer to the gold nanoparticles changes the diameter to about 40 nm. This is expected because the PSMA aptamer size is around 6 nm, which increases the total diameter by around 12 nm. Similarly, addition of anti-PSMA antibody changes the diameter to about 44 nm, which is very similar to the value recently reported by Liu et al. (18) This experiment was repeated 5-6 times, and average values are reported here. Since the hydrodynamic diameters of conjugated gold nanoparticles are very close for A9 aptamer-conjugated gold nanoparticles and anti-PSMA antibody-conjugated gold nanoparticles, we expect both aptamers and antibodies are conjugated with popcorn-shaped gold nanoparticles.

Cell Culture and Cellular Incubation with Multifunctional Nanoparticles.

Cancer cells were grown in a 5% CO2 incubator at 37° C. using RPMI-1640 medium (ATCC, Rockville, Md.) supplemented with 10% premium fetal bovine serum (FBS) (Lonza, Walkersville, Md.) and antibiotics (10 IU/mL penicillin G and streptomycin) in 75 cm² tissue culture flasks. Before the experiments, the cells were resuspended at a concentration of 1×106 cells/mL in PBS buffer medium. An enzyme-linked immunosorbent assay kit was used to quantify PSMA in different tested cells. Our experimental results indicated that the amount of PSMA in LNCaP cells was 5.8×10⁶/cell, whereas the amount of PSMA was only 1.6×10³/cell in the case of PC3 cells, which is comparable to the reported concentration of PSMA in different cancer cell lines. (see References 24,25,30)

Different numbers of cells were then immersed into the multifunctional popcorn-shaped gold nanoparticles solution for 30 minutes at room temperature before performing the next experiment.

Surface-Enhanced Raman Spectroscopy (SERS) Probe for Targeted Sensing of Cancer Cells.

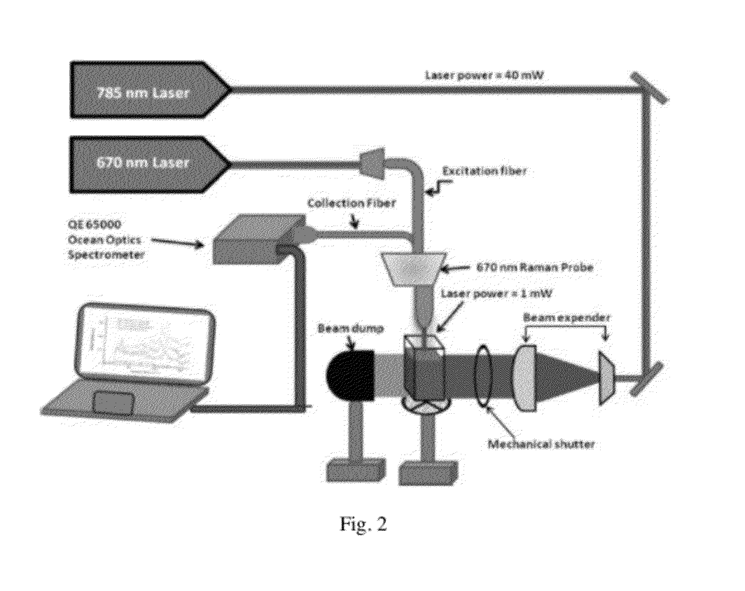

For SERS experiments, we designed a SERS probe, as described in references 39. As shown in FIG. 2, we used a continuous-wavelength DPSS laser from Laser Glow Technology (LUD-670) operating at 670 nm as an excitation light source. This light source has the capability to minimize the whole sensor configuration. We used an InPhotonics 670 nm Raman fiber optic probe for excitation and data collection. It is a combination of a 90 μm excitation fiber and a 200 μm collection fiber with filtering and steering micro-optics. We used a miniaturized QE65000 scientific-grade spectrometer from Ocean Optics as a Raman detector. The spectral response range of this mini Raman spectrometer is 220-3600 cm⁻¹. It is equipped with a TE-cooled 2048 pixel CCD and interfaced to a computer via a USB port. The Hamamatsu FFT-CCD detector used in the QE65000 instrument provides 90% quantum efficiency, with high signal-to-noise ratio and rapid signal processing speed, as well as remarkable sensitivity for low-light-level applications. The Raman spectra were collected with Ocean Optics data acquisition SpectraSuite spectroscopy software.

Photothermal Therapy and Percent of Live Cells Determination.

For photothermal therapy using NIR radiation, we used a continuous-wavelength portable OEM laser operating at 785 nm as an excitation light source for 30 minutes. After that, we performed a 3-(4,5-dimethylthiazol-2-yl)-2,5-diphenyltetrazolium bromide (MTT) test to find the amount of live cells during the nanotherapy process. For this purpose, prostate cancer cells were seeded in 96-well plates (well diameter 6.4 mm) with a density of 100,000 cells/well and allowed to attach for 24 hours at 37° C. in a 5% CO2 incubator before the treatment. Cell viability was determined 1 hour after photothermal treatment, using an MTT cell proliferation assay kit (ATCC CA no. 30-1010k). This experiment was performed 5-6 times, and average values are reported here.

Time-Dependent In Situ Photothermal Nanotherapy Study

Using SERS Probe for in situ SERS intensity measurements, we designed a portable sensor concept, as shown in FIG. 2. As discussed above, for nanotherapy we used a continuous-wavelength OEM laser operating at 785 nm as an excitation light source. For on-time SERS measurement during the therapy process, a continuous-wavelength DPSS laser from Laser Glow Technology (LUD-670) operating at 670 nm was used as an excitation light source. We used an InPhotonics 670 nm Raman fiber optic probe for excitation and data collection. It is a combination of a 90 μm excitation fiber and a 200 μm collection fiber with filtering and steering micro-optics. Both light sources have the capability to minimize the whole sensor configuration. We used a miniaturized QE65000 scientific-grade spectrometer from Ocean Optics as a SERS detector, which has remarkable sensitivity for low-light-level applications. This experiment was performed 5-6 times, and average values are reported here.

Results and Discussion of Cancer Cells

Our popcorn-shaped gold nanoparticles-based SERS approach for the selective detection of human prostate cancer cell line LNCaP is based on the fact that, in the presence of this cancer cell line, multifunctional popcorn-shaped gold nanoparticles undergo aggregation (as shown in FIG. 3B. As a result, several hot spots are formed, providing a significant enhancement of the Raman signal intensity from Rh6G-modified aptamers by several orders of magnitude (2.5×10⁹) through electromagnetic field enhancements.

Turning now to FIG. 3, it should be appreciated that FIG. 3A shows a TEM image showing anti-PSMA antibody- and A9 RNA aptamer-conjugated popcorn-shaped gold nanoparticles before the addition of cancer cell line. FIG. 3B shows a TEM image demonstrating aggregation of multifunctional popcorn-shaped gold nanoparticles after the addition of 10⁴ human prostate cancer LNCaP cells/mL for 30 min.

FIG. 3C shows a TEM image demonstrating approximately no aggregation after the addition of 10⁵ HaCaT cells/mL on multifunctional popcorn-shaped gold nanoparticles for 4 hours, where nanoparticles are randomly distributed on the whole cells.

FIG. 3D shows a TEM image showing very little or about no aggregation after the addition of 10⁵ PSMA-negative human prostate cancerous PC-3 cells/mL on multifunctional popcorn-shaped gold nanoparticles for 4 h.

FIG. 3E shows a TEM image showing the formation of cancer cell clusters in the presence of 10⁶ human prostate cancer LNCaP cells/mL.

FIG. 3F shows an absorption profile showing variation of multifunctional popcorn-shaped gold nanoparticles due to the addition of different cancerous and noncancerous cells. The strong, long-wavelength band in the visible region (λ_{PR}=580 nm) is due to the oscillation of the conduction band electrons. A new band appearing around 780 nm, due to the addition of LNCaP cells, demonstrates the aggregation of gold nanoparticles. It should be appreciated from the figures that the HaCaT noncancerous cells and PSMA-negative human prostate cancerous PC-3 cells are poorly labeled by the nanoparticles, and as a result, we do not observe any new broad band corresponding to nanoaggregates.

FIG. 3G shows a plot demonstrating SERS enhancement and selectivity of our multifunctional popcorn-shaped gold nanoparticles-based SERS assay. Raman intensity enhances 2.5×10⁹ times upon the addition of 4.8×10⁴ human prostate cancer LNCaP cells/mL, whereas Raman scattering intensity remains unchanged upon the addition of 10⁶ PC-3 cells/mL, PSMA-negative human prostate cancer cells, and 10⁶ HaCaT human skin noncancerous cells cells/mL.

FIG. 3H shows a plot demonstrating SERS scattering intensity changes upon the addition of different concentrations (number of cells/mL) of LNCaP human prostate cancer cells to multifunctional popcorn-shaped gold nanoparticle.

FIG. 3I shows a plot demonstrating how Raman intensity at 1511 cm₋₁ changes upon the addition of different concentrations (cell/mL) of LNCaP prostate cancer cell.

In the LNCaP cell line, a cancer cell has many surface epidermal growth factor prostate specific membrane antigen receptors available for specific recognition with monoclonal anti-PSMA antibody- and A9 aptamer-conjugated popcorn-shaped gold nanoparticles. As a result, after the addition of the LNCaP cell line, several nanoparticles can bind to PSMA receptors in one cancer cell, thereby producing nanoparticle aggregates (as shown in FIG. 1). Our TEM image (FIG. 3B) shows clearly that multifunctional nanoparticles are strong aggregates on the surface of cancer cells. As a result, a new broad band appears around 180 nm, far from their plasmon absorption band, as shown in FIG. 3F. This red shift might be due to two factors. One is the change in the local refractive index on the nanoparticle surface caused by specific binding of the multifunctional nanoparticle, which binds to PSMA on the LNCaP cell surface. The other is the interparticle interaction resulting from the assembly of nanoparticles on the cell surface.11-40 As shown in FIGS. 3C and 3D, our TEM image also clearly demonstrates that the HaCaT noncancerous cells and PSMA-negative human prostate cancerous PC-3 cells are poorly labeled by the multifunctional nanoparticles, even after 4 hours of incubation, and, as a result, we did not observe any new broad band corresponding to nanoaggregates (as shown in FIG. 3F). As will be appreciated from FIGS. 3C and 3D, some popcorn-shaped gold nanoparticles are also found on the HaCaT noncancerous cells and PC-3 cancerous cells, mostly due to nonspecific interactions, and thus the nanoparticles are randomly distributed on the whole cells.

The largest Raman scattering enhancements, even single molecule SERS, have been described for molecules residing in the fractal space between aggregated colloidal nanoparticles. (See references 31-39) This is attributed to plasmonic coupling between nanoparticles in close proximity, which results in huge local electromagnetic field enhancements in these confined junctions or SERS “hot spots.” As our data clearly show, cancer cell helps to generate hot spots through aggregation in multifunctional popcorn-shaped gold nanoparticle surface, and, as a result, we note about 8 orders of magnitude enhancement of Raman signal (as an excitation light source) for 30 minutes.

After that, we performed a 3-(4,5-dimethylthiazol-2-yl)-2,5-diphenyltetrazolium bromide (MTT) test to find the amount of live cells during the nanotherapy process. For this purpose, prostate cancer cells were seeded in 96-well plates (well diameter 6.4 mm) with a density of 100,000 cells/well and allowed to attach for 24 hours at 37° C. in a 5% CO2 incubator before the treatment. Cell viability was determined 1 hour after photothermal treatment, using an MTT cell proliferation assay kit (ATCC CA no. 30-1010k). This experiment was performed 5-6 times, and average values are reported here.

Time-Dependent In Situ Photothermal Nanotherapy Study Using SERS Probe.

For in situ SERS intensity measurements, we designed a portable sensor concept, as shown in FIG. 2. As discussed before, for nanotherapy we used a continuous-wavelength OEM laser operating at 785 nm as an excitation light source. For on-time SERS measurement during the therapy process, a continuous-wavelength DPSS laser from Laser Glow Technology (LUD-670) operating at 670 nm was used as an excitation light source. We used an InPhotonics 670 nm Raman fiber optic probe for excitation and data collection. It is a combination of a 90 μm excitation fiber and a 200 μm collection fiber with filtering and steering micro-optics. Both light sources have the capability to minimize the whole sensor configuration. We used a miniaturized QE65000 scientific-grade spectrometer from Ocean Optics as a SERS detector, which has remarkable sensitivity for low-light-level applications. This experiment was performed 5 to 6 times, and average values are reported here.

Results and Discussion

Our popcorn-shaped gold nanoparticles-based SERS approach for the selective detection of human prostate cancer cell line LNCaP is based on the fact that, in the presence of this cancer cell line, multifunctional popcorn-shaped gold nanoparticles undergo aggregation (as shown in FIG. 3B). As a result, several hot spots are formed, providing a significant enhancement of the Raman signal intensity from Rh6G-modified aptamers by several orders of magnitude (2.5×10⁹) through electromagnetic field enhancements. In the LNCaP cell line, a cancer cell has many surface epidermal growth factor prostate-specific membrane antigen receptors available for specific recognition with monoclonal anti-PSMA antibody- and A9 aptamer-conjugated popcorn-shaped gold nanoparticles. As a result, after the addition of the LNCaP cell line, several nanoparticles can bind to PSMA receptors in one cancer cell, thereby producing nanoparticle aggregates (as shown in FIG. 1). Our TEM image (FIG. 3B) shows clearly that multifunctional nanoparticles are strong aggregates on the surface of cancer cells. As a result, a new broad band appears around 180 nm, far from their plasmon absorption band, as shown in FIG. 3F. This red shift might be due to two factors. One is the change in the local refractive index on the nanoparticle surface caused by specific binding of the multifunctional nanoparticle, which binds to PSMA on the LNCaP cell surface. The other is the interparticle interaction resulting from the assembly of nanoparticles on the cell surface. As shown in FIG. 3C and FIG. 3D, our TEM image also clearly demonstrates that the HaCaT noncancerous cells and PSMA-negative human prostate cancerous PC-3 cells are poorly labeled by the multifunctional nanoparticles, even after 4 hours of incubation, and, as a result, we did not observe any new broad band corresponding to nanoaggregates (as shown in FIG. 3F). As noted from FIG. 3C and FIG. 3D, some popcorn-shaped gold nanoparticles are also found on the HaCaT noncancerous cells and PC-3 cancerous cells, mostly due to nonspecific interactions, and, thus, the nanoparticles are randomly distributed on the whole cells.

The largest Raman scattering enhancements, even single molecule SERS, have been described for molecules residing in the fractal space between aggregated colloidal nanoparticles (see References 31-39). This is attributed to plasmonic coupling between nanoparticles in close proximity, which results in huge local electromagnetic field enhancements in these confined junctions or SERS “hot spots.” As our data clearly show, cancer cell helps to generate hot spots through aggregation in multifunctional popcorn-shaped gold nanoparticle surface, and, as a result, we note about 8 orders of magnitude enhancement of Raman signal (as shown in FIG. 3G). The Raman modes at 236, 252, 273, and 376 cm⁻¹ are N—C—C bending modes of the ethylamine group of the Rh6G ring, and the Raman modes at 615, 778, 1181, 1349, 1366, 1511, 1570, 1603, and 1650 cm⁻¹ are due to C—C—C ring in-plane bending, C—H out-of-plane bending, C—N stretching, and C—C stretching, as previously reported (see References 39,43).

As shown in FIG. 3E, at higher concentrations of cancer cells, not only do multifunctional nanoparticles aggregate on cancer cells, but they also help for the formation of cancer cell clusters. To understand how the SERS assay responds to a single biofunctional nanoparticle, we also performed experiments on the addition of LNCaP cancer cells with only A9 aptamerconjugated popcorn-shaped gold nanoparticles, only anti-PSMA antibody-conjugated popcorn-shaped gold nanoparticles, and multifunctional nanoparticles. Our experimental results show that SERS enhancement is 4×10⁶ times for only A9 aptamerconjugated popcorn-shaped gold nanoparticles, 1.5×10⁷ times for only anti-PSMA antibody-conjugated popcorn-shaped gold nanoparticles, and 2.5×10⁹ times in the presence of multifunctional gold nanoparticles, which indicates that, for early cancer detection, multifunctional gold nanoparticles will be the best choice.

As shown in FIG. 3E, at higher concentrations of cells, multifunctional nanoparticles not only aggregate on cancer cells but also help in the formation of cancer cell clusters as the concentration of cells increases. The formation of cancer cell clusters is attributed to the fact that the aptamer- and anti-PSMA antibody-conjugated popcorn-shaped gold nanoparticles act as binding agents in between the cells to form a cellular network. When antibody from one side of the nanoparticle binds to a specific binding site on the surface of the target cancer cell, there are other antibodies on the other side of the nanoparticle that could be unoccupied, thereby providing an opportunity to bind to more than one cancer cell on each nanoparticle. Nanoparticle aggregates, which make bridges between two cells, offer an opportunity to generate perfect hot spot for SERS enhancement.

The Raman enhancement, G, is measured experimentally by direct comparison as shown below (see references 31-39).

G=(I_{SERS}/I_{Raman})(M_{bulk}/M_{ads})

where I_{SERS} is the intensity of a 1511 cm⁻¹ vibrational mode in the surface-enhanced spectrum in the presence of cancer cells, I_{Raman} is the intensity of the same mode in the bulk Raman spectrum from only Rh6G, M_{bulk} is the number of molecules in the bulk, and M_{ads} is the number of molecules adsorbed and sampled on the SERS-active substrate. All spectra are normalized for integration time. The enhancement factor estimated from the SERS signal and normal Raman signal ratio for the 1511 cm⁻¹ band is approximately 2.5×10⁹. No significant changes in Raman frequencies are observed in comparison to the corresponding SERS and Raman bands.

To evaluate whether our assay is highly selective, we also studied how SERS intensity changes upon the addition of HaCaT noncancerous cells and PSMA-negative human prostate cancer cell line (PC-3). As shown in FIG. 3G, SERS intensity was negligible in the presence of 10⁵ HaCaT cells or when we added 10⁵ PC-3 prostate cancer cells to multifunctional popcorn-shaped gold nanoparticles. Since both HaCaT and PC-3 cells do not over-express PSMA, there will be a weak interaction between multifunctional popcorn-shaped gold nanoparticles and the HaCaT or PC-3 cancer cell line. Due to the lack of strong interaction, nanoparticles do not produce enough hot spots, and, as a result, Raman enhancement is not observed. This contrast difference clearly demonstrates that our multifunctional popcorn-shaped gold nanoparticles-based SERS scattering assay is highly specific for the LNCaP human prostate cancer cell line and can even distinguish between different prostate cancer cell lines.

To evaluate the sensitivity of our SERS probe, different concentrations of LNCaP human prostate cancer cell line from one stock solution were evaluated. As shown in FIG. 3H and FIG. 3I, the SERS intensity is highly sensitive to the concentration of LNCaP human prostate cancer cells. Our experimental results (FIG. 3H) clearly demonstrate that the sensitivity of our SERS probe is as low as 50 cells. As shown in FIG. 3I, our experimental results also demonstrate that, as we increase the concentration of LNCaP cancer cell above 10⁵, the SERS intensity remains almost unchanged. This may be due to the fact that, as we increase the concentration of LNCaP cancer cells, aggregates or cluster size increases. Since it is known that small clusters are better for hot spot formation, increasing cluster size may decrease the number of hot spots, and, as a result, intensity decreases after a certain concentration of LNCaP human prostate cancer cells.

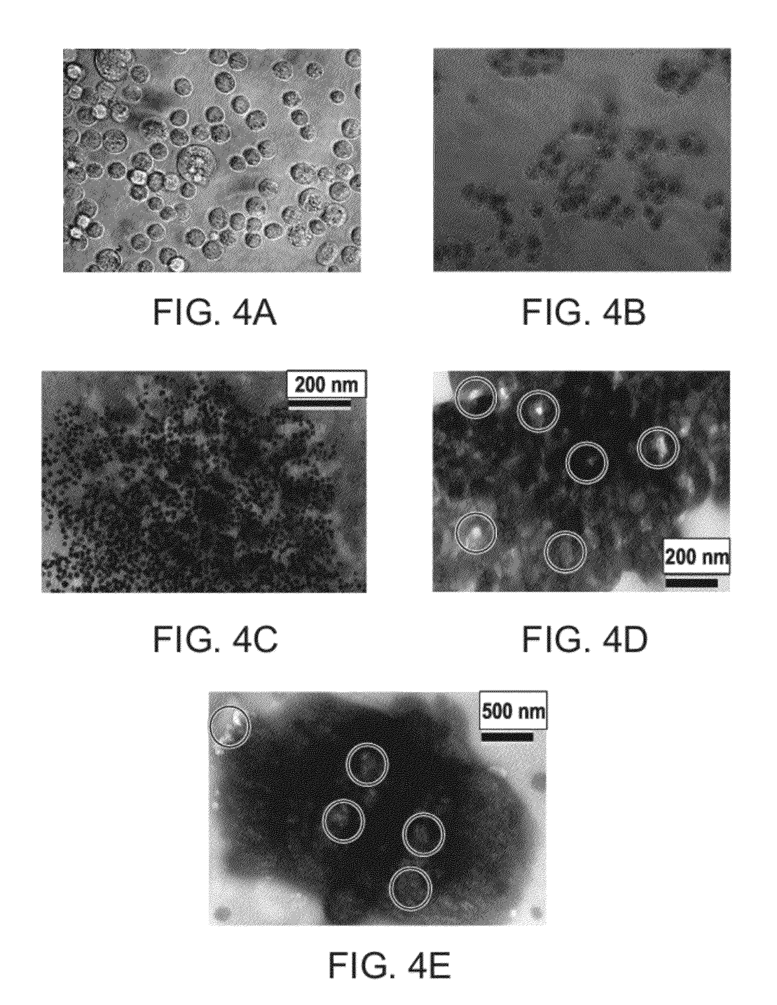

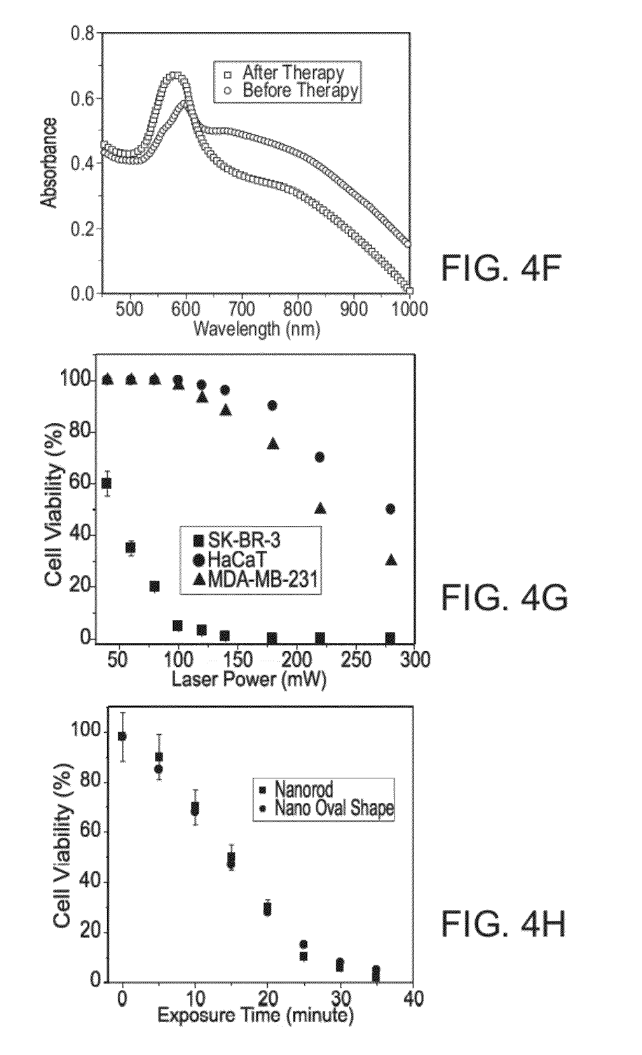

After successful targeted sensing of LNCaP human prostate cancer cells using multifunctional popcorn-shaped gold nanoparticles, we performed NIR irradiation experiments to determine whether it can be used for photothermal nanotherapy of LNCaP cancer cells. During photothermal therapy, the light absorbed by the gold nanoparticles is transferred to the antibody, aptamer, and cell environment by rapid electron-phonon relaxation in the nanoparticles, followed by phonon-phonon relaxation. In our nanotherapy experiment, we applied 80-120 mW, 785 nm NIR light for 30 minutes using a 785 nm OEM laser. This irradiation wavelength matches with the plasmon bands of the LNCaP cancer cell-conjugated popcorn-shaped gold nanoparticles. As shown in FIG. 4, exposure to the 785 nm light at 100 mW (12.5 W/cm²) caused photodestruction of whole prostate cancer cells.

FIG. 4 depicts several images and graphs of the effects on cancer cells. FIG. 4A and FIG. 4B are bright-field inverted microscope images of multifunctional popcorn-shaped gold nanoparticle-conjugated LNCaP prostate cancer cells (A) before therapy and (B) after therapy for 30 min and after being stained with Trypan Blue. FIG. 4C is a TEM image showing deformation of nano-popcorn structure after popcorn-shaped gold nanoparticle-conjugated LNCaP cells were exposed to 100 mW, 785 nm NIR continuous-wave radiation for 10 min. FIG. 4D is a TEM image showing structure deformation and irreparable damage of cancer cell surfaces after 20 min of radiation; purple circles show bubble formation. FIG. 4E is a TEM image demonstrating irreparable damage of cancer cell surfaces when multifunctional popcorn-shaped gold nanoparticle-conjugated LNCaP cells were exposed to 100 mW, 785 nm NIR continuous-wave radiation for 30 min; purple circles show bubble formation. FIG. 4F is an absorption profile demonstrating nanoparticle structural changes after nanotherapy process. FIG. 4G is a plot showing cell viability measured by MTT test after popcorn-shaped gold nanoparticle-conjugated LNCaP cells, PC-3 cells, and HaCaT cells were exposed to 785 nm NIR continuous-wave radiation at different power doses. FIG. 4H is a plot comparing photothermal therapy response between well-characterized gold nanorods and popcorn-shaped gold nanoparticles when multifunctional nanoparticle-conjugated LNCaP cells were exposed to 100 mW, 785 nm NIR continuous-wave radiation for different times.

TEM pictures (FIG. 4D and FIG. 4E) of the irradiated cells clearly show areas of massive, irreparable cell membrane disruption. After therapy, cell viability is detected by MTT test, as well as using bright-field inverted microscopy technique. To find the extent of cell death, we added Trypan Blue. FIG. 4A clearly shows that cells are 100% alive without therapy. On the other hand, FIG. 4B clearly shows that most of the cancer cells are dead after 30 minutes of the nanotherapy process. Our bright-field inverted microscope image also clearly shows that cancer cells are deformed during the nanotherapy process. This cell death following nanoparticles exposure to NIR radiation could be due to numerous factors, including nanoparticle explosion, shock waves, bubble formation, and thermal disintegration.

As shown in FIG. 4D and FIG. 4E, bubbles clearly formed (indicated by purple circles) when multifunctional popcorn-shaped gold nanoparticle-conjugated LNCaP cells were exposed to 100 mW, 785 nm NIR continuous-wave radiation. Temperature measurement indicates that the temperature increases to 48° C. during the nanotherapy process.

As shown in FIG. 4H, our time interval MTT test indicates that, within 30 minutes, most of the cancer cells died. We also performed the same tests with PSMA-negative prostate cancerous (PC-3) and noncancerous (HaCaT) cells (as shown in FIG. 4G), and we found that the cancer cells required less than half the laser energy (8 W/cm²) for photothermal lysis in comparison to the normal cells (20 W/cm²). Next, to understand how good the photothermal performance is with our popcorn-shaped gold nanoparticles, we performed the same photothermal experiment with multifunctional gold nanorods (σ=2.3) using 100 mW, 785 nm NIR continuous-wave radiation. We maintained the concentration of gold nanomaterials in such a way that, after aggregation in the presence of LNCaP cells, the absorption coefficient at 785 nm was around 0.5. FIG. 4H clearly shows that photothermal response for nano-popcorn-based gold nanoparticles is slightly better than or comparable to that of well-studied gold nanorods. Though their photothermal responses are similar, in nano-popcorn, several narrow, nano-scale corners are capable of focusing the field at their apexes, which will provide huge enhancement of SERS signal, and, as a result, nano-popcorn will be a better SERS substrate than gold nanorods.

The electron-phonon relaxation process is size and shape independent and results in temperature increases on the order of a few tens of degrees. Depending on the amount of heat generated during the photothermal process, several subsequent processes can occur: (1) The lattice cools off by passing its heat to the surrounding medium via phonon-phonon relaxation, which produces sufficient heat for the destruction of chemically attached cancer cells. (2) The lattice heat content is sufficient to lead to particle structural changes, such as nanoparticle fragmentation. (3) Due to electronic coupling of the surface gold-sulfur bond vibrations with the nanoparticle surface phonons, there is a possibility of the gold-sulfur bond breaking. As a result, dye-modified aptamers are released from the surface.

The first process will lead to irreversible cell destruction, through protein denaturation and coagulation, as well as cell membrane destruction. As shown in FIG. 4D and FIG. 4E, bubble formation around gold nanoparticles occurs during the nanotherapy process, which may impose mechanical stress, resulting in cell damage.

During the second process, there is a possibility of nanoparticle structural change. To understand whether that is occurring in this case, we performed time-dependent TEM study and absorption spectral measurements during the nanotherapy process. As shown in FIG. 4C, after 10 minutes of photothermal process, there is a clear deformation of the nano-popcorn structure. Our data clearly show that the tips have mostly vanished, and since the tips focus the field at their apexes, which provide sufficient field for SERS enhancement, SERS intensity should decrease for this deformation process. Our TEM data after 20 and 30 minutes of the nanotherapy process show huge, irreparable damage of cancer cell surfaces and bubble formation, and, as a result, it is very difficult to study the fate of the nanostructure by TEM. To get a clear picture, we recorded absorption spectra before and after the photothermal therapy process. As shown in FIG. 3F, the aggregated nanoparticle concentration decreases after therapy, and we see a strong broad band between 500 and 600 nm, which is mainly due to the deformed nanostructure formed during the therapy process. Based upon this SERS intensity change due to the thermal deformation of gold nanostructure during the photothermal process, nanotherapy response can be monitored.

During the third process, there is a possibility of breaking the gold-sulfur bond. As a result, dye-modified aptamers are released from the surface. Due to this release, the separation distance between the gold nanoparticles and Rh6G dye changes abruptly, and this destroys the SERS signal from Rh6G.

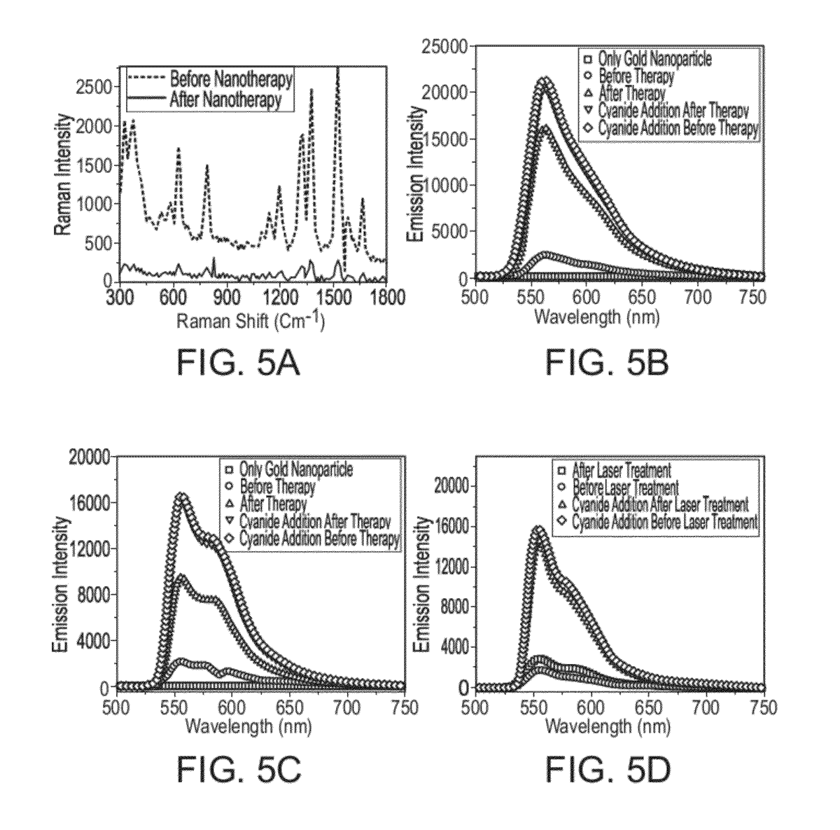

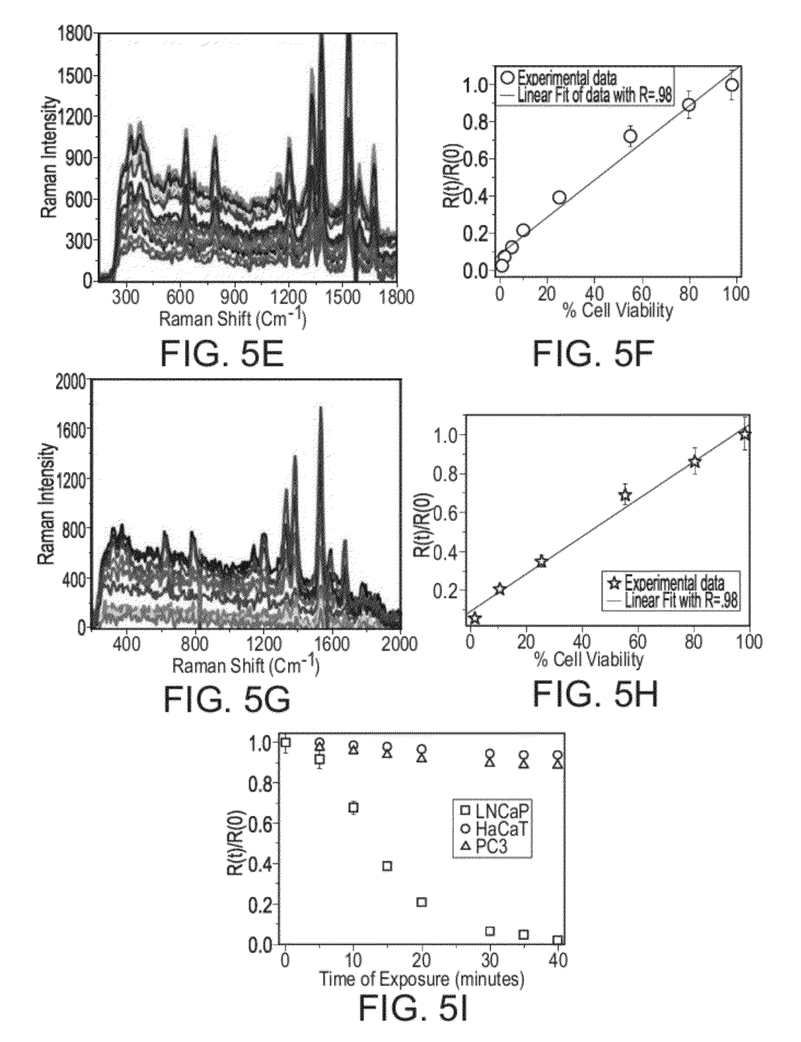

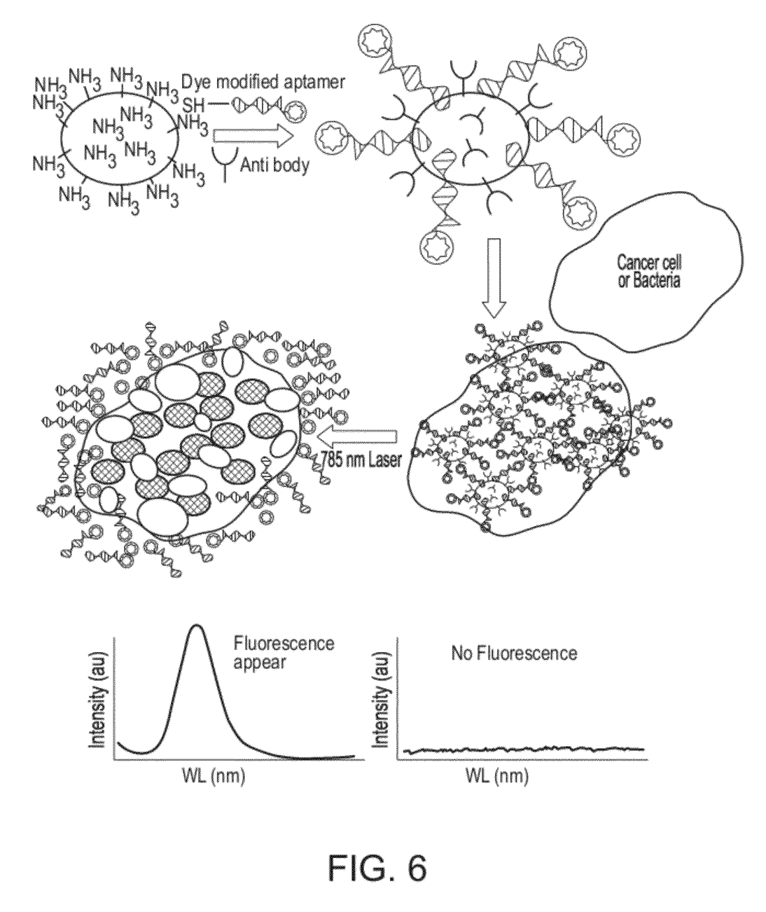

FIG. 5 shows several plots as described herein: FIG. 5A: Surface-enhanced Raman spectral change from Rh6G-modified A9 aptamer- and anti-PSMA antibody-coated popcorn-shaped gold nanoparticle conjugated LNCaP cells, before and after nanotherapy. FIG. 5B: NSET intensity change before and after photothermal therapy, when Cy3-modified A9 aptamer and anti-PSMA antibody-coated popcorn-shaped gold nanoparticle-conjugated LNCaP cells were exposed to 100 mW, 785 nm NIR continuous-wave radiation for 30 min. The plot also demonstrates how NSET intensity changes due to the addition of cyanide in both cases before and after therapy, which destroys the gold nanostructure completely. Similarly, the plot also shows that, when only Cy3 dye (100 nM) was adsorbed on gold nanoparticles, the fluorescence signal was quenched about 100%. FIG. 5C: NSET intensity change before and after photothermal therapy, when Cy3-modified anti-PSMA antibody- and A9 aptamer-coated popcorn-shaped gold nanoparticle-conjugated LNCaP cells were exposed to 100 mW, 785 nm NIR continuous-wave radiation for 30 min. The plot also demonstrates how NSET intensity changes due to the addition of cyanide in both cases before and after therapy, which destroys the gold nanostructure completely. Similarly, the plot also shows that, when only Cy3 dye (100 nM) was adsorbed onto gold nanoparticles, the fluorescence signal was quenched by almost 100%. FIG. 5D: NSET intensity change before and after laser treatment, when Cy3-modified A9 aptamer- and anti-PSMA antibody coated popcorn-shaped gold nanoparticles were exposed to 100 mW, 785 nm NIR continuous-wave radiation for 30 min. The plot also demonstrates how NSET intensity changes due to the addition of cyanide ion both cases before and after laser treatment, which destroys the gold nanostructure completely. FIG. 5E: Plot demonstrating time-dependent SERS intensity decrease during nanotherapy progress of LNCaP prostate cancer cells. SERS intensity measurements were performed at 2 min intervals during the therapy process for 30 min. FIG. 5F: Plot showing linear relationship between % of LNCaP cell viability and SERS intensity change when multifunctional popcorn-shaped gold nanoparticle-conjugated LNCaP cells were exposed to 100 mW, 785 nm NIR continuous-wave radiation for 30 min. FIG. 5G: Plot demonstrating time-dependent SERS intensity decrease during nanotherapy progress for SK-BR-3 breast cancer cell line when monoclonal anti-HER2/c-erb-2 antibody- and Cy3-modified S6 aptamer-conjugated popcorn-shaped gold nanoparticles were attached on the SK-BR-3 cell line. SERS intensity measurements were performed at 2 min intervals during the therapy process for 30 min. FIG. 5H: Plot showing linear relationship between % of SK-BR-3 cell viability and SERS intensity change when multifunctional popcorn-shaped gold nanoparticle-conjugated SK-BR-3 cells were exposed to 100 mW, 785 nm NIR continuous-wave radiation for 30 min. FIG. 5I: Plot demonstrating SERS intensity change when multifunctional popcorn-shaped gold nanoparticle-conjugated LNCaP, PC-3, and HaCaT cells were exposed to different power 785 nm NIR continuous-wave radiation for 30 min.

As shown in FIG. 5A, we observed a very distinct SERS intensity change after 30 minutes of photothermal therapy. This distinct SERS intensity change can be mainly due to two factors: (1) change in the distance between gold nanoparticles and Rh-6G dye during nanotherapy and (2) change in the morphology of the gold nanoparticles.

To understand whether gold nanoparticle structural changes and dye-modified aptamer release from the surface during nanotherapy are possible mechanisms, we also performed nanoparticle surface energy transfer (NSET) experiments using fluorescence dye Cy3-labeled A9 aptamer- and anti-PSMA antibody-attached popcorn-shaped gold nanoparticles attached to LNCaP cells for nanotherapy. (Details of the NSET experimental setup are described in references 42,44, and 47.) In this case, we used 532 nm excitation to monitor the NSET intensity change during the therapy process. As shown in FIG. 5B, when only Cy3 dye (100 nM) was adsorbed on the gold nanoparticles, the fluorescence signal was quenched about 100%. In this condition, since the acceptor nanoparticles and donor organic dyes are brought into proximity, there is dipole-surfacetype energy transfer from dye molecular dipoles to the nanometal surface, which is known as NSET. Since before therapy the Cy3-labeled A9 aptamers are attached to gold nanoparticles, the fluorescence from Cy3-labeled aptamers is more than 80% quenched by the gold nanoparticles, and, as a result, we observe very little fluorescence signal (FIG. 5B). In this condition, since the acceptor nanoparticles and donor organic dyes are about 6 nm apart, there is very strong dipole-surface-type energy transfer from dye molecular dipoles to the nanometal surface, and, as a result, more than 80% of the fluorescence was quenched. If, during therapy, dye-modified aptamers are released from the surface and gold nanoparticles, this distance change will generate a huge fluorescence signal.

Our experimental results (FIG. 5B) clearly show that NSET signal was mostly restored after nanotherapy, which confirms that the distance between the nanosurface and dye changes during the therapy process, mainly due to breaking the Au—S bond. Recently, Poon et al. also reported photothermal release of DNA through Au—S bond breaking, which also confirms that Au—S bond breaking can be a possible mechanism.

To understand whether all dye-attached aptamers are released during the therapy process, after the therapy process we treated the aptamer-conjugated gold nanoparticle-attached cancer cells with 100 μM potassium cyanide to oxidize the gold nanoparticles and release all the dye molecules into the solution. As shown in FIG. 5B, the fluorescence signal increased only 20% after the addition of cyanide, which indicates that, after therapy, a nanoparticle has only around 80 dye-attached aptamers. Our experimental result clearly shows that the third process can be a possible path for the decrease in SERS intensity during nanotherapy. As we have discussed before, a multifunctional gold nanoparticle has 400-500 dye-attached aptamers. During the conjugation of gold nanoparticles and the cancer cell line, most of the aptamers will be used for conjugation with cancer cells and some of them will be free. We believe that, during the therapy process, since the cancer cell membrane is damaged, aptamers which are conjugated to cancer cells will be removed easily. As a result, we see 80% dye released during the therapy process. FIG. 5B also shows that the fluorescence signal due to the addition of 100 μM potassium cyanide before therapy and after therapy is very similar, which clearly shows that there is no photobleaching of Cy3 dyes during the therapy process

Next, to understand whether antibody is also released during the therapy process, we performed NSET experiments using Cy3-labeled anti-PSMA antibody- and A9 aptamer-coated popcorn-shaped gold nanoparticle-attached LNCaP cell nanotherapy experiment. As shown in FIG. 5C, our experimental results clearly show that NSET signal is mostly restored after nanotherapy, which confirms that the distance between the nanosurface and dye-attached antibody changes during the therapy process. To understand how many antibodies are released during the therapy process, after therapy we treated the antibody-conjugated gold nanoparticle-attached cancer cells with 100 μM potassium cyanide. As shown in FIG. 5C, our result clearly shows that the fluorescence signal increases by only 40% after addition of cyanide, which indicates that around 60% of the antibodies are released during the therapy process. FIG. 5C also shows that the fluorescence signals due to the addition of 100 μM potassium cyanide before therapy and after therapy are very similar, which clearly shows that there is no photobleaching of Cy3 dyes during the therapy process.

After that, to understand whether the cell is necessary to release aptamers during laser irradiation in our experimental condition, we exposed Cy3-labeled A9 aptamer- and anti-PSMA antibody-coated popcorn-shaped gold nanoparticles to 100 mW, 785 nm NIR continuous-wave radiation for 30 minutes and monitored the time-dependent NSET intensity. As shown in FIG. 5D, our experimental results clearly show that NSET signal changes very little, which confirms that the distance between the nanosurface and dye-attached multifunctional nanoparticles does not change significantly during the therapy process. To understand how many aptamers are released during the therapy process, after therapy we treated the multifunctional nanoparticle with 100 μM potassium cyanide. As shown in FIG. 5D, our result clearly shows that the fluorescence signal increases by about 95% after addition of cyanide, which indicates that less than 5% of the aptamers are released during the laser treatment process. FIG. 5D also shows that the fluorescence signal due to the addition of 100 μM potassium cyanide before and after laser treatment is very similar, which clearly shows that there is no photobleaching of Cy3 dyes during the therapy process. The absence of Au—S bond breaking without the cancer cell line may be due to the following facts: (1) Several articles have demonstrated that antibody-assisted aggregation of Au nanoparticles on cell membranes or in intracellular environments led to a high enhancement of photothermal performance. (see references 60-63) Since in our case aggregates were formed only in the presence of cancer cells, which have a strong absorption at 785 nm, the photothermal therapy effect should be highly efficient in the presence of aggregates. (2) Multifunctional popcorn-shaped gold nanoparticles exhibit very little absorption at 785 nm.

Next, we performed in situ time-dependent SERS measurements during the nanotherapy process to understand whether our SERS assay can monitor the nanotherapy process, using SERS intensity change. For in situ SERS intensity measurements, we designed a portable sensor, as described before (FIG. 2). As we discussed before, for nanotherapy we used a continuous-wavelength OEM laser operating at 785 nm as an excitation light source. FIG. 5E demonstrates how the SERS intensity decreases during the therapy process. We recorded the data in 2 minute intervals during the therapy process. Our in situ time-dependent measurements clearly show that, as the nanotherapy progresses, the SERS intensity decreases. To understand whether there is any correlation between % of LNCaP prostate cancer cell death and SERS intensity change, we plotted % of cell viability and SERS intensity change. As shown in FIG. 5F, we see a nice linear plot between % of LNCaP prostate cancer cell viability and SERS intensity change, where R(t) is the SERS intensity at time t during nanotherapy, and R(0) is the initial SERS intensity before therapy. Our result clearly shows that it is possible to use SERS assay to measure in situ nanotherapy response during the therapy process for the LNCaP prostate cancer cell line.

To understand whether our technique is versatile, we also tested whether our SERS-based approach can be applicable to monitor the nanotherapy process for a breast cancer cell line. For this purpose, a well-characterized breast cancer cell line, SK-BR-3, which over-expresses epidermal growth factor receptor HER2/c-erb-2/Neu was used. For specific recognition of the SKBR-3 cell line, we modified popcorn-shaped gold nanoparticles with monoclonal anti-HER2/c-erb-2 antibody and Rh6G-modified S6 aptamer. For photothermal therapy, we exposed the multifunctional popcorn-shaped gold nanoparticle-attached SKBR-3 cells to 100 mW, 785 nm laser light for 30 minutes and used MTT test to find the cell viability. We also performed in situ time-dependent SERS measurements during the nanotherapy process, using the same procedure as for LNCaP prostate cancer.

As shown in FIG. 5G, our in situ time-dependent measurement clearly shows that, as the SK-BR-3 cell line nanotherapy progresses, the SERS intensity decreases, similar to what we observed for the LNCaP prostate cancer cell line. To find whether there is any correlation between % of cell death and SERS intensity change, we plotted % of SK-BR-3 cell viability and SERS intensity change. FIG. 5H shows a nice linear plot between % of SK-BR-3 breast cancer cell viability and SERS intensity change. Our result clearly demonstrates that it is possible to use SERS assay for the measurement of in situ nanotherapy response during the therapy process for different cancer cell lines.

To understand whether the SERS intensity change depends on the formation of conjugation between multifunctional gold nanoparticles and the cancer cell line, we followed the time-dependent SERS intensity change during the photothermal process for PC-3 and HACaT cell lines in the presence of Rh6Gmodified A9 aptamer- and anti-PSMA antibody-coated popcorn-shaped gold nanoparticles. As we discussed before, multifunctional gold nanoparticles were not conjugated well with PC-3 and HaCaT cell lines, and, as a result, we do not expect much nanotherapy activity for these cell lines when they were exposed to 100 mW, 785 nm laser light for 30 minutes. As shown in FIG. 5I, SERS intensity changes little in the case of PC-3 and HaCaT cell lines, whereas SERS intensity changes abruptly for the LNCaP cell line. Our data clearly demonstrates that our SERS assay for nanotherapy monitoring is highly selective and nanoparticle conjugation with cancer cells is necessary to monitor the therapy process. The SERS sensor developments will have important implications in the development of better bioassays for monitoring tumor response to therapy during photothermal nanotherapy in clinical cases.

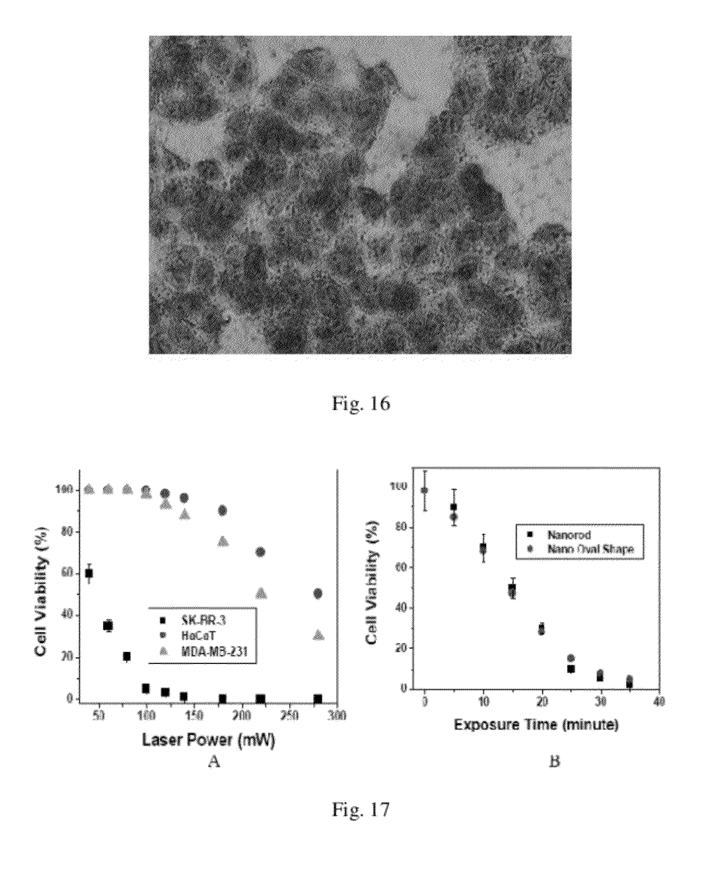

Further assays were performed to further test the efficacy of the nanotherapy. FIG. 16 shows bright field inverted microscope images of multifunctional popcorn-shaped gold nanoparticle conjugated HEPG2 liver cancer cells, after therapy for 30 minutes and after stained with Trypan Blue. Our result demonstrate that about 100% cell death after 30 minutes therapy. It should be appreciated from FIG. 16, that the therapy is very effective for this cancer as well.

Although it is clear that various cancer cells can be killed using the disclosed methods, FIG. 17A provides a plot demonstrating that popcorn-shaped gold nanotechnology base photothermal therapy can be used for selective killing of cancer cell. Cell viability was measured by MTT test after oval shape gold nanoparticle-conjugated SK-BR-3 cells, MDA-MB-231 and HaCaT cells were exposed to 785 nm NIR continuous-wave radiation at various power doses. As will be appreciated from the plot in FIG. 17A, the cell viability for the different cancer cells differs with laser power.

FIG. 17B is a plot showing a comparison of photothermal therapy response between well-characterized gold nanorod and oval shape gold nanoparticle, when multifunctional nanoparticle conjugated SK-BR-3 cells are exposed to 100 mW 785 nm NIR continuous-wave radiation for different times. It should be appreciated that both are effective on killing cell off the cancer cells.

Sensing, Targeting, and Therapy for Drug Resistance Salmonella DT104 Bacteria.

That same nanotechnology approach described above with respect to cancer cells can be used to selectively target and destroy multiple drug resistant bacteria (MDRB) Salmonella DT104 bacteria. The steps for destroying bacteria would include: (1) Synthesis of popcorn-shaped gold nanoparticles of different sizes; (2) Design of multifunctional popcorn-shaped gold nanoparticle for targeted sensing, therapy and monitoring therapy effectiveness for MDRB Salmonella DT104 bacteria; (3) Design of portable SERS sensor for MDRB Salmonella DT104 bacteria detection, treatment and for monitoring treatment effectiveness using the same instrument; and (4) detect and kill MDRB Salmonella DT104 c bacteria.

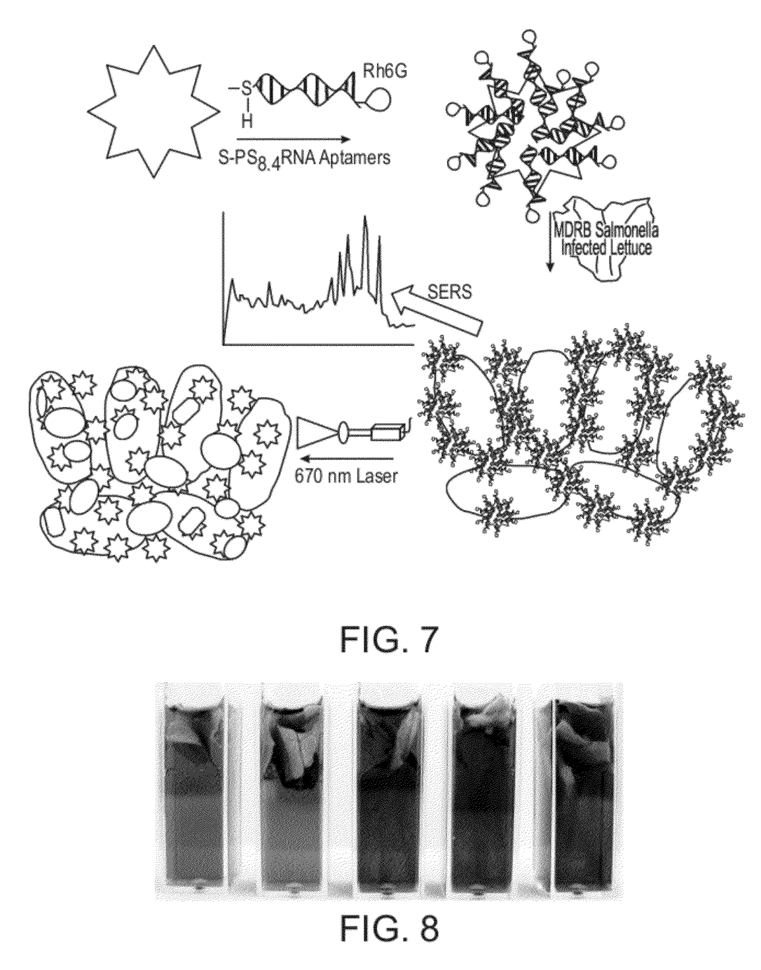

As will be appreciated from FIG. 7, infected conjugated popcorn shape S-PS8.4 RNA aptamers conjugated popcorn shape gold nanotechnology can be used to selectively detect and destroy drug resistant Salmonella bacteria from infected lettuce.

FIG. 8 shows a series of photograph showing colorimetric change upon the addition of drug resistant MDRB Salmonella typhimurium DT104 bacteria infected lettuce on the S-PS8.4 RNA aptamers-conjugated popcorn-shaped gold nanoparticles. From left to right, the vial 1 contains lettuce infected by the infected by the Salmonella typhimurium DT104 bacteria solution containing 10⁵ cfu/mg bacteria. Vial 2 contains lettuce infected by the infected by the MDRB Salmonella typhimurium DT104 bacteria solution containing 10⁴ cfu/mg bacteria. Vial 3 contains lettuce infected by the MDRB Salmonella typhimurium DT104 bacteria solution containing 10³ cfu/mg bacteria. Vial 4 contains lettuce infected by the E-Coli bacteria solution containing 10⁶ cfu/mg bacteria. Vial 5 contains normal lettuce without MDRB Salmonella typhimurium bacteria.

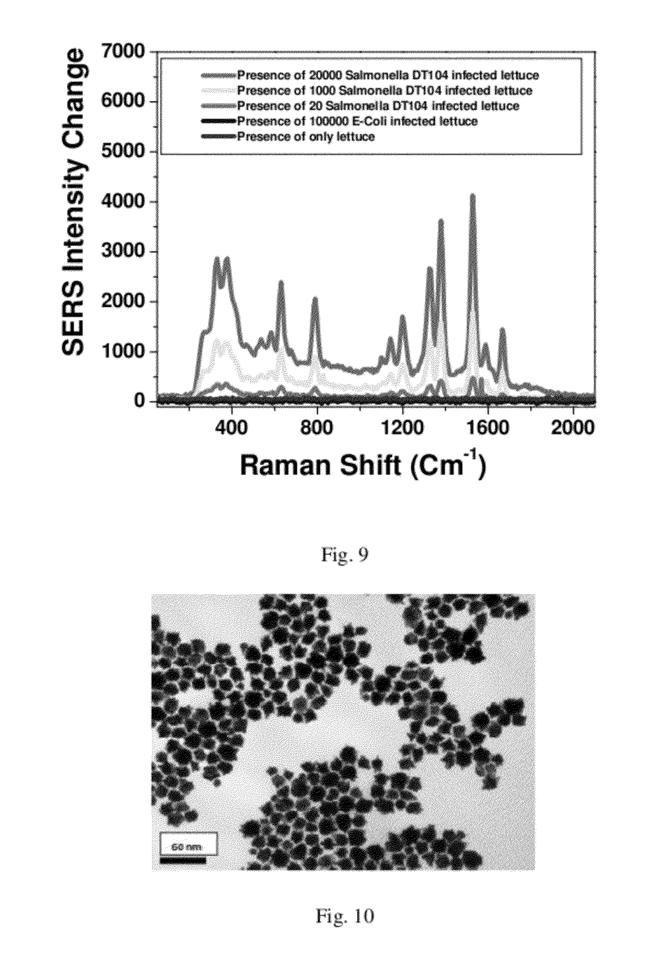

FIG. 9 shows a series of SERS intensity change upon the addition of MDRB Salmonella typhimurium bacteria infected lettuce directly on the Rh-6G modified S-PS8.4 RNA aptamers-conjugated popcorn-shaped gold nanoparticles. It also shows selectivity with respect to E-Coli bacteria and sensitivity up to 20 CFU/mg MDRB Salmonella DT104 bacteria infected lettuce.

As further evidence of the assays efficacy on pathogenic bacteria, TEM images were taken before and after addition of drug resistant MDRB Salmonella DT104 bacteria to S-PS8.4 RNA aptamers-conjugated popcorn-shaped gold nanoparticles. FIG. 10 shows a TEM image showing S-PS8.4 RNA aptamers-conjugated popcorn-shaped gold nanoparticles before the addition of MDRB Salmonella DT104 bacteria. FIG. 11 shows a TEM image demonstrating aggregation of S-PS8.4 RNA aptamers-conjugated popcorn-shaped gold nanoparticles after the addition of 10³ cfu/mL MDRB Salmonella DT104 bacteria.

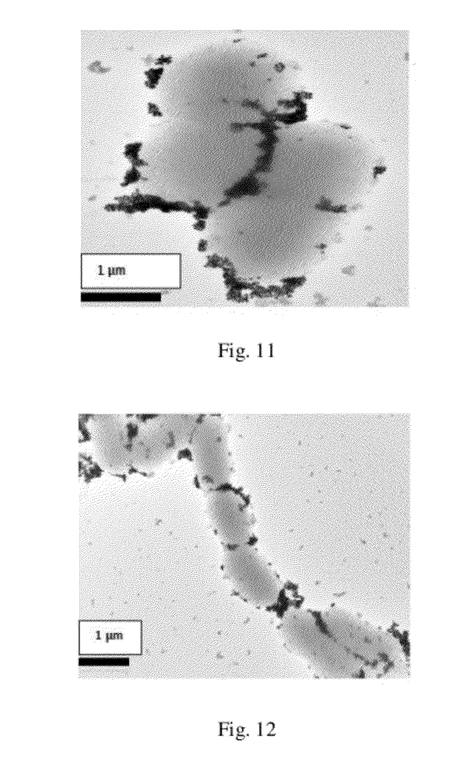

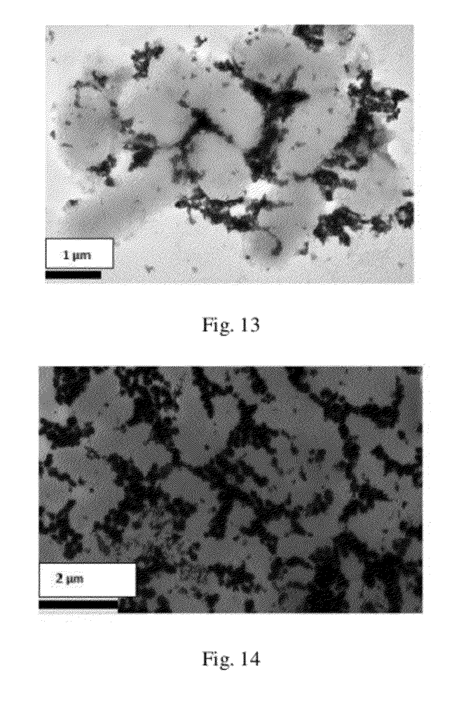

Larger concentrations of bacteria results in further aggregation and formation of microbial clusters. FIG. 12 shows TEM image demonstrating aggregation of S-PS8.4 RNA aptamers-conjugated popcorn-shaped gold nanoparticles, as well as formation of microbial clusters in presence of 10⁴ cfu/mL drug resistant Salmonella MDRB Salmonella DT104 bacteria. FIG. 13 shows TEM image demonstrating aggregation of S-PS8.4 RNA aptamers-conjugated popcorn-shape gold nanoparticles, as well as formation of microbial clusters in presence of 10⁵ cfu/mL drug resistant Salmonella typhimurium bacteria. FIG. 14 shows a TEM image demonstrating aggregation of S-PS8.4 RNA aptamers-conjugated popcorn-shaped gold nanoparticles, as well as formation of microbial clusters in presence of 10⁶ cfu/mL drug resistant Salmonella typhimurium bacteria.

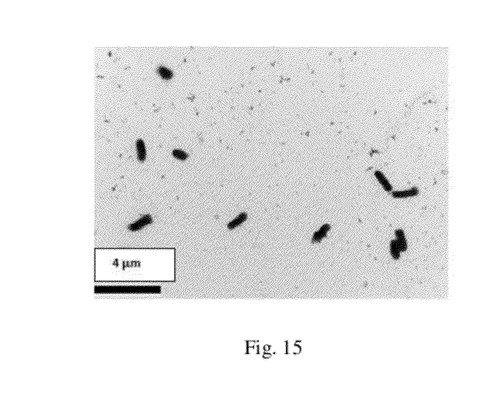

As evidence of the specificity demonstrated by our assay, we analyzed the affects of S-PS8.4 RNA aptamers-conjugated popcorn-shaped gold nanoparticles on E. coli. FIG. 15 shows a TEM image of E-Coli bacteria in presence S-PS8.4 RNA aptamers-conjugated popcorn-shaped gold nanoparticles. Our TEM image clearly demonstrates that the E-Coli bacteria cells are poorly labeled by S-PS8.4 RNA aptamers-conjugated popcorn-shaped gold nanoparticles.

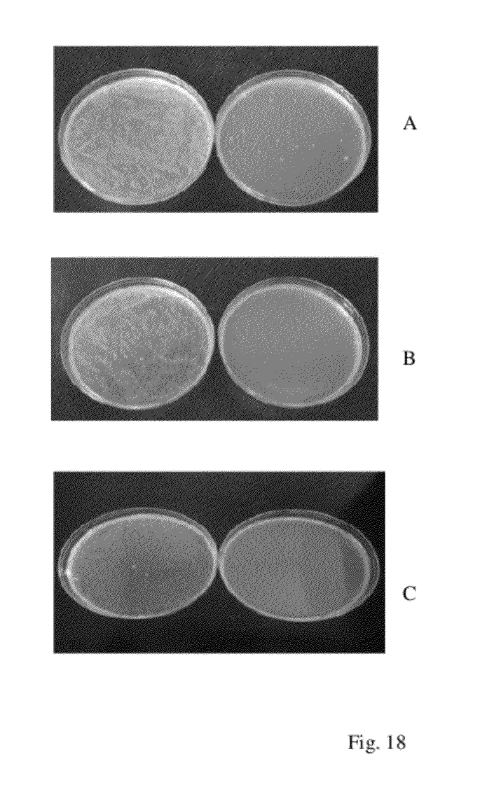

To demonstrate the effectiveness of phototherapy on pathogenic bacteria, the assay was performed on colonies of drug resistant Salmonella typhimurium. FIG. 18 show colonies of MDRB Salmonella DT104 bacteria before (left) and after (right) exposing infected lettuce with S-PS8.4 RNA aptamers-conjugated popcorn shape gold nanoparticle using 670 nm light for 20 minutes. FIG. 18A shows lettuce incubated with 10⁶ cfu/mL MDRB Salmonella DT104 bacteria. FIG. 18B shows lettuce incubated with 10⁵ cfu/mL MDRB Salmonella DT104 bacteria and FIG. 18C shows lettuce incubated with 10⁴ cfu/mL MDRB Salmonella DT104 bacteria. There clearly is a marked reduction in visible colonies following phototherapy.

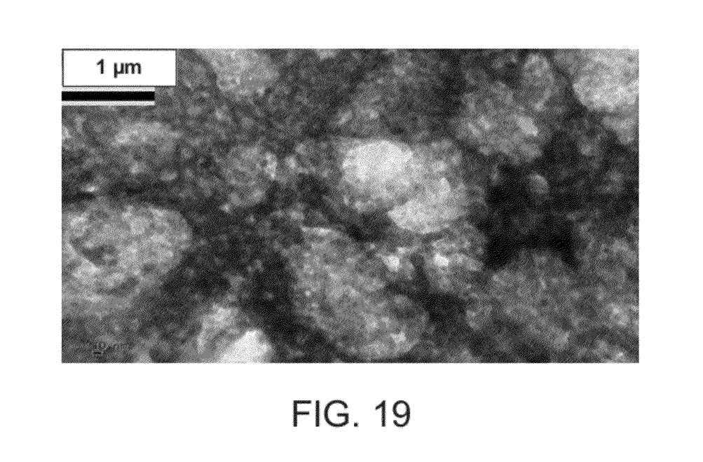

A TEM images was taken to observe the effects of the phototherapy on bacteria. FIG. 19 is a TEM image demonstrating irreparable damage of cluster of bacterial cell surfaces when S-PS8.4 RNA aptamers-conjugated popcorn shape gold nanoparticle conjugated MDRB Salmonella DT104 bacteria were exposed to 676 nm NIR radiation for 20 minutes. It is clear that the assay described above with respect to cancer cells can be adapted for use with pathogenic bacteria.

### CONCLUSION

In conclusion, we have disclosed a multifunctional popcorn-shaped gold nanotechnology-driven surface-enhanced Raman scattering assay for targeted sensing, nanotherapy treatment, and in situ monitoring of photothermal nanotherapy response during the therapy process. We have shown that, in the presence of LNCaP human prostate cancer cells, multifunctional popcorn-shaped gold nanoparticles form several hot spots and provide a significant enhancement of the Raman signal intensity from Rh6G-modified aptamers by several orders of magnitude (2.5×10⁹) through electromagnetic field enhancements. We have also shown that the same approach works in destroying pathogenic bacteria. The approach can therefore be used to destroy bacteria or cancer cells.

Our experimental data with a HaCaT noncancerous cell line, as well as with a PSMA-negative PC-3 prostate cancer cell line, clearly demonstrate that our SERS assay is highly sensitive to LNCaP and was able to distinguish it from other breast cancer cell lines. Our experiment indicates that this bioassay is highly sensitive, with a detection ability of about 50 cancer cells. We have clearly demonstrated that, when popcorn-shaped multifunctional gold nanoparticles are attached to cancerous cells, the localized heating that occurs during NIR irradiation is able to cause irreparable cellular damage. This popcorn-shaped gold nanotechnology-based assay is rapid, taking about 30 minutes from cancer cell binding to detection and destruction of the cell. Our data clearly show that the photothermal response for nano-popcorn-based gold nanoparticles is slightly better than or comparable to that for well-studied gold nanorods.

Our in situ time-dependent experimental results clearly demonstrate that, as the nanotherapy progresses, the SERS intensity decreases, and, as a result, by monitoring the SERS intensity change, one can monitor the photothermal therapy response over time. Our experimental data indicate a nice linear plot between % cancer cell death and SERS intensity change, which clearly shows that it is highly feasible to use SERS assay for the measurement of in situ nanotherapy response during the therapy process, which is critical to providing effective treatment of cancer.

After optimization of different parameters, we believe that this nanotechnology-driven assay could have enormous potential for application in rapid, on-site targeting of cancer cells and MDRB bacteria, nanotherapy treatment, and monitoring the nanotherapy process, which is critical.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### REFERENCES

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).
(1) Bray, F.; Møller, B. Nat. ReV. Cancer 2006, 6, 63-74.
(2) http://www.who.int/cancer/en.
(3) http://www.cancer.org/docroot/cri/content/cri_{—}2_{—}4_{—}1x_what_are_the_key_statistics_for_prostate_cancer_{—}36.asp.
(4) http://www.cdc.gov/cancer/prostate.
(5) http://www.cdc.gov/cancer/dcpc/data/men.htm.
(6) Shewach, D. S.; Kuchta, R. D. Chem. ReV. 2009, 109, 2859-2861.
(7) Deutscher, S. L. Chem. ReV. 2010, 110, 196-3211.
(8) Louie, A. Chem. ReV. 2010, 110, 3146-3195.
(9) Ferrari, M. Nat. ReV. Cancer 2005, 5, 161-171.
(10) Scheinberg, D. A.; Villa, C. H.; Escorcia, F. E.; McDevitt, R. M. Nat. ReV. Clin. Oncol. 2010, 7, 266-276.
(11) Lal, S.; Clare, S. E.; Halas, N. J. Acc. Chem. Res. 2008, 41, 1842-1851.
(12) Jain, P. K.; Huang, X.; El-Sayed, I. H.; El-Sayed, M. A. Acc. Chem. Res. 2008, 41, 1578-1586.
(13) Cheon, J.; Lee, J.-H. Acc. Chem. Res. 2008, 41, 1630-1640.
(14) Peer, D.; Karp, J. M.; Hong, S.; Farokhzad, O. C.; Margalit, R.; Langer, R. Nature Nanotechnol. 2007, 2, 751-760.
(15) Sarkar, B.; Dosch, J.; Simeone, D. M. Chem. ReV. 2009, 109, 3200-3208.
(16) Nam, J.; Won, N.; Jin, H.; Chung, H.; Kim, S. J. Am. Chem. Soc. 2009, 131, 13639-13645.
(17) Yu, J.; Javier, D.; Yaseen, M. A.; Nitin, N.; Richards-Kortum, R.; Anvari, B.; Wong, M. S. J. Am. Chem. Soc. 2010, 132, 1929-1938.
(18) Liu, X.; Dai, Q.; Austin, L.; Coutts, J.; Knowles, G.; Zou, J.; Chen, H.; Huo, Q. J. Am. Chem. Soc. 2008, 130, 2780-2782.
(19) Agasti, S. S.; Chompoosor, A.; Chang-Cheng, Y.; Ghosh, P.; Kim, C. K.; Rotello, V. M. J. Am. Chem. Soc. 2009, 131, 5728-5729.
(20) Sha, M. Y.; Xu, H.; Natan, M. J.; Cromer, R. J. Am. Chem. Soc. 2008, 130, 17214-17215.
(21) Lutz, B. R.; Dentinger, C. E.; Nguyen, L. N.; Sun, L.; Zhang, J.; Allen, A. N.; Chan, S.; Knudsen, B. S. ACS Nano 2008, 2, 2306-2314.
(22) Stoeva, S. I.; Lee, J.-S.; Smith, J. E.; Rosen, S. T.; Mirkin, C. A. J. Am. Chem. Soc. 2006, 128, 8378-8379.
(23) Huang, X.; El-Sayed, I. H.; Qian, W.; El-Sayed, M. A. J. Am. Chem. Soc. 2006, 128, 2115-2120
(24) Javier, D. J.; Nitin, N.; Levy, M.; Ellington, A.; Richards-Kortum, R. Bioconjugate Chem. 2008, 19, 1309-1312.
(25) Sardana, G.; Jung, K.; Stephan, C.; Diamandis, E. P. J. Proteome Res. 2008, 7, 3329-3338.
(26) Lu, W.; Arumugam, S. A.; Senapati, D.; Singh, A. K.; Arbneshi, T.; Khan, S. A.; Yu, H.; Ray, P. C. ACS Nano 2010, 4, 1739-1749.
(27) Huang, Y.-F.; Liu, H.; Xiong, X.; Chen, Y.; Tan, W. J. Am. Chem. Soc. 2009, 131, 17328-17334.
(28) Qian, X.; Zhou, X.; Nie, S. J. Am. Chem. Soc. 2008, 130, 14934-14935.
(29) Jain, P. K.; Qian, W.; El-Sayed, M. A. J. Am. Chem. Soc. 2006, 128, 2426-2433.
(30) Gordon, I. 0.; Tretiakova, M. S.; Noffsinger, A. E.; Hart, J.; Reuter, V. E.; Ahmadie, H. A. Mod. Pathol. 2008, 21, 1421-1427.
(31) Brown, S. D.; Nativo, P.; Smith, J. A.; Stirling, D.; Edwards, P. R.; Venugopal, B.; Flint, D. J.; Plumb, J. A.; Graham, D.; Wheate, N. J. J. Am. Chem. Soc. 2010, 132, 4678-4684.
(32) Camden, J. P.; Dieringer, J. A.; Zhao, J.; Van Duyne, R. P. Acc. Chem. Res. 2008, 41, 1653-1661.
(33) Brus, L. Acc. Chem. Res. 2008, 41, 1742-1749.
(34) Moskovits, M. ReV. Mod. Phys. 1985, 57 3 783 826.
(35) Camden, J. A.; Dieringer, J. A.; Wang, Y.; Masiello, D. J.; Marks, L. D.; Schatz, G. C.; Van Duyne, R. P. J. Am. Chem. Soc. 2008, 130, 12616-12617.
(36) Barhoumi, A.; Zhang, D.; Tam, F.; Halas, N. J. J. Am. Chem. Soc. 2008, 130, 5523-5529.
(37) Laurence, T. A.; Braun, G.; Talley, C.; Schwartzberg, A.; Moskovits, M.; Reich, N.; Huser, T. J. Am. Chem. Soc. 2009, 131, 162-169.
(38) Bonham, A. J.; Braun, G.; Pavel, I.; Moskovits, M.; Reich, N. O. J. Am. Chem. Soc. 2007, 129, 14572-14573.
(39) Dasary, S. S. R.; Singh, A. K.; Senapati, D.; Yu, K.; Ray, P. C. J. Am. Chem. Soc. 2009, 131, 13806-13812.
(40) Wang, S.; Singh, A. K.; Senapati, D.; Neely, A.; Yu, H.; Ray, P. C. Chem. Eur. J. 2010, 16, 5600-5606.
(41) Darbha, G. K.; Singh, A. K.; Rai, U.S.; Yu, E.; Yu, H.; Ray, P. C. J. Am. Chem. Soc. 2008, 130, 8038.
(42) Griffin, J.; Singh, A. K.; Senapati, D.; Rhodes, P.; Mitchell, K.; Robinson, B.; Yu, E.; Ray, P. C. Chem. Eur. J. 2009, 15, 342-351.
(43) Tiwari, V.; Tovmachenko, O.; Darbha, G. K.; Hardy, W.; Singh, J. P.; Ray, P. C. Chem. Phys. Lett. 2007, 446, 77-82.
(44) Griffin, J.; Ray, P. C. J. Phys. Chem. B 2008, 112, 11198-11201.
(45) Yun, C. S.; Javier, A.; Jennings, T.; Fisher, M.; Hira, S.; Peterson, S.; Hopkins, B.; Reich, N. 0.; Strouse, G. F. J. Am. Chem. Soc. 2005, 127, 3115-3119.
(46) Skewis, L. R.; Reinhard, B. M. Nano Lett. 2008, 8, 214-220.
(47) Darbha, G. K.; Ray, A.; Ray, P. C. ACS Nano 2007, 3, 208-214.
(48) Neely, A.; Perry, C.; Varisli, B.; Singh, A. K.; Arbneshi, T.; Senapati, D.; Kalluri, J. K.; Ray, P. C. ACS Nano 2009, 3, 2834-2840.
(49) Mallouk, T. E.; Yang, P. J. Am. Chem. Soc. 2009, 131, 7937-7939.
(50) Singh, A. K.; Senapati, D.; Wang, S.; Griffin, J.; Neely, A.; Candice, P.; Naylor, K. M.; Varisli, B.; Kalluri, J. R.; Ray, P. C. ACS Nano 2009, 3, 1906-1912.
(51) Lorenzo, L. R.; Javier, F.; Abajo, G.; Liz-Marzn, L. M. J. Phys. Chem. C 2010, 114, 7336-7340.
(52) Khoury, C. G.; Vo-Dinh, T. J. Phys. Chem. C 2008, 112, 18849-18859.
(53) Huang, Y. F.; Chang, H. T.; Tan, W. Anal. Chem. 2008, 80, 567-572.
(54) Lilja, H.; Ulmert, D.; Vickers, A. J. Nat. ReV. Cancer 2008, 8, 268-278.
(55) Chikezie, O.; Madu, Y. L. J. Cancer 2010, 1, 150-177.
(56) Nikoobakht, B.; Wang, J.; El-Sayed, M. A. Chem. Phys. Lett. 2002, 366, 17-23.
(57) Orendorff, C. J.; Murphy, C. J. J. Phys. Chem. B 2006, 110, 3990-3994. Poon, L.; Zandberg, W.; Hsiao, D.; Erno, Z.; Sen, D.; Gates, B. D.; Branda, N. R. ACS Nano 2010, 4, 6395-6403.
(59) Wijaya, A.; Schaffer, S. B.; Pallares, I. G.; Schifferli, K. H. ACS Nano 2009, 3, 80-86.
(60) Lapotko, D.; Lukianova, E.; Potapnev, M.; Aleinikova, O.; Oraevsky, A. Cancer Lett. 2006, 239, 36.
(61) Govorov, A. O.; Richardson, H. H. Nano Today 2007, 2, 30.
(62) Richardson, H. H.; Carlson, M. T.; Tandler, P. J.; Hernandez, P.; Govorov, A. O, Nano Lett. 2009, 9, 1139.
(63) Wang, S.; Chen, K. J.; Wu, T. H.; Wang, H.; Lin, W. Y.; Ohashi, M.; Chiou, P. Y.; Tseng, H. R. Angew. Chem., Int. Ed. 2010, 49, 3777-3781.
(64) H. K. Allen, J. Donato, H. H. Wang, K. A. Cloud-Hansen, J. Davies, J. Handelsman, Call of the wild: antibiotic resistance genes in natural environments, Nat. Rev. Microbiol., 2010, 8, 251-259
(65) D. I. Andersson and D. Hughes, Antibiotic resistance and its cost: is it possible to reverse resistance? Nat. Rev. 2010, 8, 260-271.
(66) http://www.who.int/cancer/en
(67) http://www.cancer.gov/cancertopics/types/breast
(68) http://www.cancer.gov/cancertopics/types/prostate
(69) http://www.cdc.gov/cancer/nbccedp
(70) http://www.cancer.org/docroot/home/index.asp
(71) http://en.wikipedia.org/wiki/Multiple_drug_resistance
(72) http://www.who.int/tb/challenges/mdr/en/index.html
(73) Anant K. Singh, Wentong Lu, Dulal Senapati, Sadia Afrin Khan, Zhen Fan, Tapas Senapati, Teresa Demeritte, Lule Beqa, and Paresh Chandra Ray, Small, 2011, 7, 2517-2525
(74) Sadia Afrin Khan, Anant K. Singh, Dulal Senapati, Zhen Fan and Paresh C Ray, Chem. Commun., 2011, 47, 9444-9446.
(75) W Lu, A K Singh, S A Khan, D Senapati, H Yu and P C Ray, Gold Nano-Popcorn Based Targeted Diagonosis, Nanotherapy Treatment and In-Situ Monitoring of Photothermal Therapy Response of Prostate Cancer Cells Using Surface Enhanced Raman Spectroscopy, J. Am. Chem. Soc., 2010, 132, 18103-18114.
(76) Sadia Afrin Khan, Anant K. Singh, Dulal Senapati, Zhen Fan and Paresh Chandra Ray, J. Mater. Chem., 2011, 21, 17705-17709.

I claim:

1. A method for detecting human cancer cells comprising:
a) synthesizing popcorn-shaped gold nanoparticles of different sizes;
b) conjugating said popcorn-shaped gold nanoparticle with aptamers or antibodies specific to said human cancer cells; and
c) performing surface enhanced Raman spectroscopy on said location of cells.

2. The method of claim 1 wherein said human cancer cells are liver, breast, skin, or prostate.

3. The method of claim 1 wherein said aptamers are A9 RNA anti-PSMA aptamers attached to Raman dye (Rh6G).

4. A method for killing human cancer cells comprising the steps of:
a) synthesizing popcorn-shaped gold nanoparticles of different sizes;
b) conjugating said popcorn-shaped gold nanoparticle with aptamers or antibodies specific to said human cancer cells;
c) performing surface enhanced Raman spectroscopy on said location of cells; and
d) exciting said location of said human cancer cells using light.

5. The method of claim 2 wherein said light is laser light.

6. The method of claim 4 wherein said human cancer cells are liver, breast, skin, or prostate.

7. A method for detecting multiple drug resistant bacteria comprising:
a) synthesizing popcorn-shaped gold nanoparticles of different sizes;
b) conjugating said popcorn-shaped gold nanoparticle with aptamers or antibodies specific to said bacteria; and
c) performing surface enhanced Raman spectroscopy on said location of bacteria.

8. The method of claim 7 wherein said bacteria are of the species Salmonella typhimurium.

9. The method of claim 7 wherein said aptamers are A9 RNA anti-PSMA aptamers attached to Raman dye (Rh6G).

10. A method for killing bacteria comprising the steps of:
a) synthesizing popcorn-shaped gold nanoparticles of different sizes;
b) conjugating said popcorn-shaped gold nanoparticle with aptamers or antibodies specific to said bacteria;
c) performing surface enhanced Raman spectroscopy on said location of said bacteria; and
d) exciting said location of said bacteria using light.

11. The method of claim 10 wherein said light is laser light.

12. The method of claim 10 wherein said bacteria is of the species Salmonella typhimurium.

13. The method of claim 10 wherein the steps are performed in vivo.

14. The method of claim 10 wherein the steps are performed on food.
